(19) United States
(12) Patent Application Publication
(10) Pub. No.: US 2019/0185869 A1
(43) Pub. Date: Jun. 20, 2019

(54) P. SOMNIFERUM PLANTS FOR THE PRODUCTION OF CODEINE

(71) Applicant: TASMANIAN ALKALOIDS PTY LTD, Westbury, Tasmania (AU)

(72) Inventors: Gregory Michael SYMONS, Turners Marsh, Tasmania (AU); Corey James HUDSON, South Launceston, Tasmania (AU); Mark Leon LIVERMORE, Mayfield, Tasmania (AU)

(21) Appl. No.: 16/329,937

(22) PCT Filed: Sep. 4, 2017
(86) PCT No.: PCT/AU2017/050960

§ 371 Date: Mar. 1, 2019

(30) Foreign Application Priority Data
Sep. 2, 2016 (AU) 2016222511
Sep. 2, 2016 (AU) 2016903543

(51) Int. Cl. C12N 15/82

(52) U.S. Cl. CPC C12N 15/8243; C12N 15/825

(57) ABSTRACT

There are provided plants of Papaver somniferum for the production of codeine, methods for providing the plants, and poppy straw, concentrate of poppy straw, latex, opium and codeine from the plants. The plants have codeine as the predominant alkaloid. Also disclosed are plants in which a stably inheritable high codeine chemotype trait is linked to a recessive trait for lighter leaf colour. Methods of producing such plants through the exposure of parent plant lines to mutagenizing agents are also disclosed.

### FIELD OF THE INVENTION

The present invention relates to plants of Papaver somniferum for the production of codeine, and to poppy straw and latex from the plants.

### BACKGROUND OF THE INVENTION

Codeine is an opiate used for its analgesic, antitussive and antidiarrheal properties. It is by far the most widely used opiate in the world and very likely the most commonly used drug overall according to numerous reports over the years by organizations such as the World Health Organization.

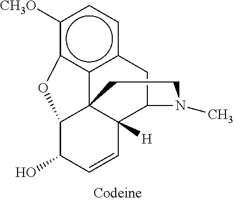

Codeine is a natural alkaloid of the opium poppy plant Papaver somniferum L. but normally constitutes only a minor fraction of the total alkaloids e.g., typically only 5-20% of the level of morphine. While codeine can be extracted from opium and poppy straw, the demand for codeine far exceeds the current available natural supply so most of the codeine currently being manufactured (85-90 per cent) is obtained from morphine through the process of O-methylation. The major part of the world's licit morphine production is to support the manufacture of codeine.

Codeine is used to manufacture Active Pharmaceutical Ingredients (APIs) such as codeine phosphate, codeine sulfate, codeine hydrochloride and codeine base, which are in turn is used to manufacture e.g., high-volume, over-the-counter, dosage forms for the relief of pain (analgesics) and cough (antitussives). Codeine is also the starting material and prototype of a large class of mainly mild to moderately strong opioids such as dihydrocodeine and hydrocodone and its derivatives such as nicocodeine and oxycodone. Thus catalytic hydrogenation of codeine yields dihydrocodeine (used to manufacture APIs such as dihydrocodeine tartrate) that can be converted by Oppenauer oxidation to dihydrocodeinone (hydrocodone, used to manufacture APIs such as hydrocodone bitartrate).

The industrial-scale methylation of morphine, at the phenolic hydroxyl group at position 3, to give codeine is usually conducted using quaternary ammonium methylating agents, typically trimethylphenylammonium chloride, in the presence of various bases such as alkali hydroxides, alkoxides, or carbonates/bicarbonates. Use of the quaternary ammonium methylating agent greatly diminishes the competing methylation of the alkaloid tertiary amine function which is a major problem with other methylating agents such as methyl halides or dimethyl sulfate, where the formation of the alkaloid quaternary salts causes loss of yield and the generation of other impurities such as alpha- and beta-codimethine via the Hofmann elimination reaction. However this solution to the quaternisation problem comes at the cost of generating the objectionally-odiferous and toxic byproduct N,N-dimethylaniline (DMA) which must be completely removed from the product and imposes waste disposal, occupational health and safety (OHS) and environmental concerns.

Most methylation procedures using trimethylphenylammonium reagents in essence involve the exposure of the trimethylammonium morphinate ion pair to high temperatures (exceeding 90 deg C.) in a non-polar, water-immiscible solvent such as toluene or xylene, where the ion pair rapidly collapses to codeine and DMA. The necessity for such solvents creates further OHS and environmental burdens on the manufacture and imposes costs associated with solvent recovery and purification. Under the vigorous conditions necessary, it is imperative that the stoichiometry of the reagents be carefully controlled to avoid on the one hand, undermethylation, leaving too much unreacted morphine, or on the other hand overmethylation, leading to formation of codeine-O(6)-methyl ether (“methylcodeine”). Both situations lead to yield losses both directly, through lower codeine formation, and indirectly through further codeine losses attending the removal of the unreacted morphine or the methycodeine impurities. Morphine is relatively easy to remove e.g., by washing a toluene solution of the codeine with aqueous alkali but this process also sacrifices some codeine to the washes, while methylcodeine is difficult to remove and may require extensive processing to achieve desired limits. Another problematic impurity created by the synthesis from morphine is dimethylpseudomorphine, created by methylation of a common impurity in Concentrate of Poppy Straw Morphine (CPS-M), pseudomorphine (2,2′-bismorphine). Dimethylpseudomorphine is particularly difficult to remove from codeine and imposes high yield losses due to the additional processing required.

The manufacture of codeine from morphine requires extensive processing at some stage to remove colour bodies originating either in opium or in the CPS-M); in the latter case the colour bodies derive from the initial extraction process that produces morphine from poppy straw. The extraction conditions required to recover morphine from poppy straw are such that there is considerable, unavoidable extraction of coloured materials from the poppy and the CPS-M may typically have several per cent of non-alkaloid material. With opium as input, the raw material is grossly impure (e.g., 5-24% morphine) and it is mandatory to purify and separate the morphine from the other alkaloids and colour bodies before input to codeine manufacture, while with CPS-M, some manufacturers upgrade the morphine to technical quality before manufacture of codeine, while others enter the material directly into the methylation and then process to remove colour post-methylation. However, both approaches have considerable cost penalties associated with the additional processing, inevitable yield losses, capacity and opportunity costs.

Codeine is more soluble than morphine in virtually all common media and is consequently easier to extract from poppies and easier to purify. Hence, the yield, quality and cost of natural codeine obtained from a codeine poppy are all improved relative to synthetic codeine obtained from natural morphine.

Alkaloids are extracted from the poppy capsules of Papaver somniferum by two commercial methods. In one method, the immature capsule is cut and the latex collected from the wound. The air-dried latex is opium which, according to the Merck Index, 11th Edition, contains alkaloids in the amounts shown in Table I. In the second method, the mature poppy capsules and the poppy capsule stems are collected, and threshed to remove the seeds and form a straw. When necessary, the straw is dried to a water content below 16%. Solvent or water extraction is employed to remove the alkaloids from the straw.

However, the yield of codeine is confounded with that of other alkaloids and a poppy producing a higher content of codeine relative to impurity alkaloids such as e.g., thebaine and oripavine would enable a simpler extraction/purification process, resulting in higher yields, better quality and throughput and lower costs.

Where solvent or water or super critical fluid, such as CO₂, extraction is employed to remove the alkaloids from the straw, such method, as practiced, involves the production of “Concentrate of Poppy Straw”. Concentrate of Poppy Straw (or “CPS”) is described as “The material arising when poppy straw has entered into a process for the concentration of its alkaloids, when such material is made available in trade,” (Multilingual dictionary of narcotic drugs and psychotropic substances under international control, United Nations, New York, 1983). Not inconsistent with the foregoing description, Concentrate of Poppy Straw is described as “the crude extract of poppy straw in either liquid, solid or powder form which contains the phenanthrene alkaloids of the opium poppy, 45 U.S. Federal Register 77466, Nov. 24, 1980”. When in liquid form, the liquid is preferably concentrated before entering into commerce. The generally preferred Concentrate of Poppy Straw is the powder form which results from removing the solvent or water following extraction of the poppy straw. According to the United Nations publication ‘Narcotic Drugs: Estimated World Requirements for 2007; Statistics for 2005 (E/INCB/2006/2)’, “Concentrate of Poppy Straw is the dried residue obtained through the extraction of alkaloids from poppy straw. Until the second half of the 1990s, only concentrate of poppy straw containing morphine as the main alkaloid was manufactured. Since then, concentrate of poppy straw containing mainly thebaine or oripavine has started to be manufactured.”

Some relatively small quantities of CPS codeine are produced in a few countries as a by-product of CPS morphine extraction. These quantities are not significant in world trade.

The production of codeine by the cultivation of non-morphine-containing poppies greatly decreases the risk the potential for diversion and abuse of morphine. Papaver somniferum poppies grown in the field and which yield a poppy straw containing codeine as the predominant alkaloid and little or no morphine are described in International Patent Publication No. WO 2009/143574 in the name of Tasmanian Alkaloids Pty Ltd. The codeine content in the straw of those poppies is at least 40% by weight or greater of the alkaloid combination of morphine, codeine, thebaine and oripavine, and plants are described that produce straw containing codeine in an amount up to 90% by weight or greater of the above noted alkaloid combination with the absolute codeine content of the straw ranging from about 0.8% to about 4.2% on a dry weight basis of the straw. The thebaine content of the straw is also substantially reduced.

Whilst the Papaver somniferum plants of WO 2009/143574 represent a major advance in the production of codeine, further reducing the amount of thebaine and/or other confounding alkaloids present in the poppy straw, latex and opium from such “codeine plants” would markedly increase the efficiency of codeine extraction and the yield of codeine obtained.

### SUMMARY OF THE INVENTION

The present invention in one or more forms relates to the provision of plants of Papaver somniferum having a high codeine chemotype for the production of codeine wherein absolute codeine content is increased and/or wherein the amount of at least thebaine as a confounding alkaloid is reduced relative to codeine, compared to the plants from which they were derived. By reducing the level of at least thebaine in poppy straw and/or latex relative to codeine the efficiency with which codeine is extracted from the poppy straw and latex (or concentrate of poppy straw or opium) can be increased. In particularly preferred embodiments, the parent plant, from which a plant embodied by the invention is derived, is a high codeine producing Papaver somniferum of the type described in WO 2009/143574. That an increase in absolute codeine content and a decrease in thebaine content relative to codeine can be obtained in such plants is surprising given the relative level of thebaine in those plants was already low. Further, as thebaine is an intermediate compound in the synthesis of codeine in Papaver somniferum, it would always be expected to be present in their poppy straw or latex. Hence, a reduction in the thebaine content relative to codeine together with an increase in absolute codeine content in plants described herein indicates that the efficiency with which codeine is synthesized in the plants is increased and/or that plant regulatory mechanism(s) have been modified compared to the parent plant(s).

In providing the invention, the inventors have also found that particularly high levels of codeine and/or codeine relative to thebaine can be provided by plants embodied by the invention having a substantially lighter leaf colour than Papaver somniferum with a codeine chemotype conventionally grown for the codeine production. Despite the substantially lighter leaf colour the plants were found to exhibit good vigour and to be suitable for use as a field crop. This is also surprising as such plants would normally be expected to have low vigour, as light leaf colour is suggestive of a serious defect such as low leaf chlorophyll or a severe nutrient deficiency, which would manifest as poor growth rates with consequential low capsule number, poor straw and alkaloid content and/or yield. Typically, when seeking to develop high alkaloid producing Papaver somniferum , such plants would be discarded at an early stage of the program once the lightness of the leaf colour had become apparent. However, in contradiction to this, the inventors cultivated the plants to maturity and further investigated their alkaloid profile leading to the unexpected finding that the plants can nevertheless exhibit excellent codeine content with relatively low thebaine contamination despite the light leaf colour of the plants. Thus, in at least some embodiments of the invention, the light leaf colour may act as a biomarker for one or more of increased codeine content in poppy straw and/or poppy capsule latex, and increased codeine content relative to at least thebaine in the poppy straw or latex/opium.

In particular, in an aspect of the invention there is provided an isolated plant of Papaver somniferum derived from a parent plant and having a stably heritable codeine chemotype in which codeine is the predominant alkaloid, or a descendent plant thereof, wherein the codeine content of the plant is such that upon the harvesting of its poppy capsules the plant will yield a poppy straw having a higher codeine content by weight and a lower thebaine to codeine ratio than the parent plant.

Typically, the chemotype of the plant is characterised by higher codeine content by weight than a combined morphine and thebaine content.

Typically, the plant is a mutant plant derived from the parent plant. In another aspect of the invention there is provided an isolated mutant plant of Papaver somniferum derived from a parent plant and having a stably heritable codeine chemotype characterised by higher codeine content by weight than a combined morphine and thebaine content, or a descendent plant thereof, wherein the codeine content of the mutated plant is such that upon the harvesting of its poppy capsules the plant will yield a poppy straw having a higher codeine content by weight and/or a lower thebaine to codeine ratio than the parent plant.

In another aspect of the invention there is provided a method for producing a plant of Papaver somniferum having a stably heritable codeine chemotype characterised by higher codeine content than morphine and thebaine content, or a descendent plant thereof, the method comprising the steps of:

a) exposing at least one poppy seed of a Papaver somniferum parent plant to a mutagenizing agent, the parent plant being a plant which upon the harvesting of its poppy capsules will yield a poppy straw having codeine constituting about 40% by weight or greater of an alkaloid combination comprising morphine, codeine, thebaine and oripavine;

b) growing the at least one poppy seed exposed to the mutagenizing agent to produce one or more further plants, optionally through one or more self-fertilised generations; and

c) providing a plant identified from the one or more plants in step b) to be a plant which upon the harvesting of its poppy capsules will yield a poppy straw having a higher absolute codeine content and a lower thebaine to codeine ratio, than the poppy straw of the parent plant.

In another aspect of the invention there is a method for providing increased codeine content in poppy straw of a plant of Papaver somniferum relative to at least thebaine in the poppy straw, the method comprising the steps of:

a) providing at least one poppy seed of a Papaver somniferum parent plant, the parent plant being a plant which upon the harvesting of its poppy capsules will yield a poppy straw having codeine constituting about 40% by weight or greater of an alkaloid combination comprising morphine, codeine, thebaine and oripavine;

b) exposing the at least one poppy seed to a mutagenizing agent;

c) growing the at least one poppy seed exposed to the mutagenizing agent to produce one or more further plants, optionally through one or more self-fertilised generations; and

d) providing a plant identified from the one or more further plants produced in step c) to be a plant which upon the harvesting of its poppy capsules will yield a poppy straw having a higher absolute codeine content by weight and a lower thebaine to codeine ratio, than the poppy straw of the parent plant.

Typically, the parent plant has a blockage in an alkaloid synthesis pathway inhibiting or preventing the conversion of thebaine to oripavine in the parent plant. Typically also, the blockage in the alkaloid synthesis pathway is stably inherited in a plant embodied by the invention.

Typically, the parent plant contains substantially no oripavine in the poppy straw from the plant. Most typically, there is substantially no morphine and substantially no oripavine in the alkaloid combination of the parent plant utilised in a method embodied by the invention.

Typically, in plants embodied by the invention, there is substantially no morphine in the poppy straw of the plant and most usually, substantially no morphine and substantially no oripavine in the poppy straw.

In another aspect of the invention there is provided a plant of Papaver somniferum, wherein the plant is a plant of NCIMB 42630, or a descendent plant thereof.

In another aspect of the invention there is provided a plant of Papaver somniferum produced by a method embodied by the invention, or a descendent plant thereof.

In at least some embodiments a plant in accordance with the invention will have a trait for leaf colour whereby the leaves of the plant are predominantly a colour that is visually lighter relative to the leaves of the parent plant.

Typically, the colour of the leaves of these plants will predominantly be in the green-yellow colour spectrum and exhibit a dominant wavelength value of from about 561 nm or greater as determined by reflective spectrophotometry of the adaxial leaf surface using D65 illumination. Most typically, the dominant wavelength value will be in a range of from about 561 nm to about 568 nm.

Typically, the colour of the leaves of the parent plant as described herein from which a plant embodied by the invention is derived will also be in the green-yellow colour spectrum.

Typically, the lighter leaf colour of a plant embodied by the invention is controlled by a single, recessive gene.

Typically, the poppy straw of a plant embodied by the invention will have a thebaine content relative to codeine of about 25% or less on a w/w basis.

Typically, the poppy straw of a plant embodied by the invention will have a codeine content of at least about 0.832% on a w/w basis. Most typically, the codeine content of the poppy straw is about 2.5% or greater on a w/w basis.

Typically, there is substantially no morphine or oripavine in the poppy straw of a plant embodied by the invention.

In another aspect of the invention there is provided a plant of Papaver somniferum having a stably heritable codeine chemotype in which codeine is the predominant alkaloid, or a descendent plant thereof, wherein the codeine chemotype of the plant is such that upon the harvesting of its poppy capsules, the plant will yield a poppy straw having a codeine content by weight of about 2.5% w/w or greater and a thebaine to codeine ratio of about 25% w/w or less, and wherein the plant has a trait for leaf colour whereby the leaves of the plant are predominantly of a colour exhibiting a dominant wavelength in a range of from about 561 nm to about 568 nm as determined by reflective spectrophotometry of the adaxial leaf surface using D65 illumination.

In another aspect of the invention there is provided a plant of Papaver somniferum having a stably heritable codeine chemotype in which codeine is the predominant alkaloid, or a descendent plant thereof, wherein the codeine chemotype of the plant is such that upon the harvesting of its poppy capsules, the plant will yield a poppy straw having a thebaine to codeine ratio of about 25% w/w or less, and wherein the plant has a trait for leaf colour whereby the leaves of the plant are predominantly of a colour exhibiting a dominant wavelength in a range of from about 561 nm to about 568 nm as determined by reflective spectrophotometry of the adaxial leaf surface using D65 illumination.

In another aspect of the invention there is provided an isolated plant of Papaver somniferum having a stably heritable codeine chemotype in which codeine is the dominant alkaloid, wherein the plant has a trait for a leaf colour phenotype associated with a reduced leaf pigment content and one or both of a higher codeine content by weight and/or a lower thebaine to codeine ratio by weight in poppy straw yielded by harvested mature poppy capsules of the plant.

In another aspect of the invention there is provided an isolated plant of Papaver somniferum having a stably heritable codeine chemotype in which codeine is the dominant alkaloid, wherein the plant has a single-gene recessive trait for a light leaf colour phenotype associated with one or both of a higher codeine content by weight and/or a lower thebaine to codeine ratio by weight in poppy straw yielded by harvested mature poppy capsules of the plant.

In another aspect of the invention there is provided an isolated viable plant of Papaver somniferum having a stably heritable codeine chemotype in which codeine is the predominant alkaloid, wherein the leaves of the plant are predominantly of a colour exhibiting a dominant wavelength in a range of from about 561 nm to about 568 nm as determined by reflective spectrophotometry on the adaxial leaf surface using D65 illumination.

In another aspect of the invention there is provided an asexually produced viable Papaver somniferum having a stably heritable codeine chemotype in which codeine is the predominant alkaloid, wherein the P. somniferum has a leaf exhibiting a dominant wavelength in a range of from about 561 nm to about 568 nm as determined by reflective spectrophotometry on the adaxial surface of the leaf using D65 illumination.

In another aspect of the invention there is provided a poppy straw obtained at least partially from a Papaver somniferum having a stably heritable codeine chemotype in which codeine is the predominant alkaloid, the P. somniferum having a leaf exhibiting a dominant wavelength in a range of from about 561 nm to about 568 nm as determined by reflective spectrophotometry on an adaxial surface of the leaf using D65 illumination.

In another aspect of the invention there is provided a method for the production of codeine which comprises the steps of: a) harvesting mature poppy capsules of a plant embodied by the invention or a descendent plant thereof, to produce a poppy straw; and b) extracting the codeine from the poppy straw.

In still another aspect of the invention there is provided a method for the production of codeine which comprises the steps of: a) collecting and drying the latex of immature poppy capsules of a plant embodied by the invention or a descendent plant thereof to produce opium; and b) extracting the codeine from the opium.

In another aspect there is provided a plant of Papaver somniferum produced by a method embodied by the invention, or a descendent plant thereof.

In another aspect there is provided a stand of plants embodied by the invention.

In another aspect there is provided a poppy straw obtained from a plant embodied by the invention, or from a descendent plant thereof.

In another aspect there is provided a concentrate of poppy straw being a concentrate of poppy straw of a plant embodied by the invention, or from a descendent plant thereof.

In another aspect there is provided a latex for the extraction of codeine being a latex from immature poppy capsules of a plant embodied by the invention, or from immature poppy capsules of a descendent plant thereof.

In another aspect there is provided an opium for the extraction of codeine comprising an opium obtained by the drying of latex from immature poppy capsules of a plant embodied by the invention, or of a descendent plant thereof.

In another aspect there is provided a codeine extracted from the poppy straw, concentrate of poppy straw, latex or opium embodied by the invention.

In still further aspects there is provided a plant cell or plant root of a plant embodied by the invention, or of a descendent plant thereof.

One of ordinary skill in the art will understand that the total alkaloid content in the poppy straw, concentrate of poppy straw, latex or opium in embodiments of the present invention will total (but will not exceed) 100%.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers, integers or steps.

Any discussion of documents, acts, materials, devices, articles or the like that has been included in this specification is solely for the purpose of providing a context for the invention. It is not to be taken as an admission that any or all of these matters form part of the prior art base or were common general knowledge in the field relevant to the invention as it existed in Australia or elsewhere before the priority date of this application.

The features and advantages of the invention will become further apparent from the following detailed description of embodiments thereof together with the accompanying drawings.

### BRIEF DESCRIPTION OF THE ACCOMPANYING DRAWINGS

FIG. 1 is a graph showing codeine and thebaine content in the mature capsules of 20 M3 Papaver somniferum mutant plant lines in a field trial (Hagley, Tasmania, Australia) conducted during the 2012/13 poppy growing season. The lines PW08-2308, PW10-0149 and PW11-4063 are ‘Tasman’ Papaver somniferum exhibiting a codeine chemotype.

FIG. 2 is a photograph showing ‘Tasman’ M3 mutant line EM4-0045 (middle foreground) during a disease resistance screening field trial (Hagley, Tasmania, Australia) conducted during the 2012/13 poppy growing season. The EM4-0045 line exhibited pale, light-green leaves compared to the parent ‘Tasman’ line (PW08-2308) from which it was derived and other Papaver somniferum lines in surrounding plots not expressing the lighter leaf colour trait of the EM4-0045 line, and was found to have exceptionally high codeine and low thebaine content in its mature capsules.

FIG. 3 is a further photograph clearly showing the substantially lighter leaf colour of field grown EM4-0045 plants (left plot) compared to the typical darker leaf colour phenotype of commercially grown ‘Tasman’ P. somniferum. In this example, the ‘Tasman’ line EM3-1217 (right plot) shown. Both lines were derived from the same ‘Tasman’ parent line, PW08-2308.

FIG. 4 is a three-dimensional plot of L*a*b* values for each of ten PW08-2308, EM4-0045 and EM3-0006 line leaf samples from planthouse grown plants.

FIG. 5 is a photograph showing two leaves each of EM4-0045 (left) and EM3-006 (right) planthouse grown plants.

### DETAILED DESCRIPTION OF EXEMPLARY EMBODIMENTS OF THE INVENTION

Codeine is an intermediate in the morphine biosynthesis pathway in Papaver somniferum. The morphine biosynthesis pathway is highly complex involving multiple alkaloid intermediates and enzymatic steps, and is but one of a number of pathways present in Papaver somniferum involving the synthesis of benzylisoquinoline alkaloids such as codeine and morphine. Whilst substantial progress has been made in elucidating the steps and identifying the intermediate alkaloids involved in these pathways, their regulation remains poorly understood as a result of the interplay of branching of the pathways combined with the involvement of as yet unraveled feedback and homeostatic mechanisms. As a result, modification of a step in these pathways can have unpredictable effects on the synthesis of other intermediates in the same or branching pathways. This is further complicated by factors such as the potential for an enzyme to have a role in the synthesis of more than one alkaloid intermediate and, for example, the tissue-specific synthesis of alkaloids. Consequently, the effect of changes introduced into alkaloid synthesis pathways of Papaver somniferum can have significant and unpredictable effects. Nevertheless, in providing the present invention the inventors have found that thebaine content can be reduced relative to codeine in Papaver somniferum plants which produce codeine as the predominant or sole alkaloid in the alkaloid combination of morphine, codeine, thebaine and oripavine, even when the amount of thebaine relative to codeine in the parent plant is already low.

Persons skilled in the art will be able to readily grow plants of Papaver somniferum embodied by the invention and reproduce them, collect latex or produce poppy straw from them, and purify codeine from the latex (or opium) or the straw as described herein.

Seed of an example of a plant of Papaver somniferum (PW08-2308) which can be utilized as the parent plant in a method embodied by the invention was deposited under the Budapest Treaty with the National Collection of Industrial, Food and Marine Bacteria (NCIMB Ltd, Ferguson Building, Craibstone Estate, Bucksburn, Aberdeeen AB21 9YA, Scotland, United Kingdom), on 24 Aug. 2016, under Accession No. NCIMB 42631. Further such plants and methods for producing them are described in International Patent Publication No. WO 2009/143574 in the name of Tasmanian Alkaloids Pty Limited, the entire contents of which are incorporated herein by cross-reference.

As one enablement of the present invention, seed of a plant of Papaver somniferum (EM4-0045) embodied by the invention was deposited under the Budapest Treaty with the National Collection of Industrial, Food and Marine Bacteria (NCIMB Ltd, Ferguson Building, Craibstone Estate, Bucksburn, Aberdeeen AB21 9YA, Scotland, United Kingdom), on 24 Aug. 2016, under Accession No. NCIMB 42630.

The availability of these seeds is not to be construed as a license to practice this invention in contravention of rights granted under the authority of any government in accordance with its patent or breeder's rights laws.

In a particularly preferred embodiment, a plant embodied by the invention can be provided by subjecting seed a Papaver somniferum parent plant as described herein to mutagenesis. The production of mutagenized seed is well known in the art. Methods of seed mutagenesis as well as mutagens suitable for use in these methods, such as, ethyl methanesulfonate (EMS), are described in the Manual on Mutation Breeding, 2nd ed., I.A.E.A., Vienna 1977 or in Plant Breeding, Principles and Prospects, Chapman and Hall, London 1993. For mutagenesis of seed by X-ray, hydrated seeds may be treated with 20,000 rads, (30 cm from the source for 45 minutes using a filter). X-ray mutagenesis is described and compared to EMS mutagenesis by Filippetti, A. et al., “Improvement of Seed Yield in Vicia Faba L. By Using Experimental Mutagenesis II Comparison of Gamma-Radiation and Ethyl-Methane-Sulphonate (EMS) in Production of Morphological Mutants”, Euphytica 35 (1986) 49-59. DEB (diepoxybutane) mutagenized seeds may, for example, be obtained by soaking the seeds in water overnight, then soaking in 22 mM DEB for 4 hours, followed by extensive washing. Other mutagens which may be utilized include ethyl-2-chloroethyl sulphide, 2-chloroethyl-dimethylamine, ethylene oxide, ethyleneimine, dimethyl sulphonate, diethyl sulphonate, propane sulphone, beta-propiolactone, diazomethane, N-methyl-N-nitrosourethane, acridine orange and sodium azide.

Mutagenesis utilizing EMS is well described in the literature. The Manual on Mutation Breeding, supra, reports a preferred EMS mutagenesis process for barley seeds as practiced by K. Mikaelson. In this preferred process, the seeds are prepared, pre-soaked, treated with the mutagen and post-washed.

U.S. Pat. No. 6,067,749, incorporated by reference herein in its entirety, describes the use of EMS for the preparation of a Papaver somniferum strain with a high concentration of thebaine and oripavine.

Irradiation methods such as fast neutron mutagenesis (“FNM”) may also be used to produce mutagenized seed (see e.g., Li, X. et al., A fast neutron deletion mutagenesis-based reverse genetics system for plants, The Plant Journal 27(3), 235-242 (2001)). Fast neutron mutagenesis is, for instance, described by Kodym and Afza (2003), Physical and Chemical Mutagenesis, pp 189-203, in Methods in Molecular Biology, Vol. 236: Plant Functional Genomics: Methods and Protocols (Ed. E. Grotewold), Humana Press Inc, Totowa, N.J.

Gamma (γ) rays are electromagnetic waves of very short wavelengths and are obtained by disintegration of radioisotopes Co or Cs. γ sources can be installed in a r cell, a γ room, or γ field. These are shielded by lead or concrete. Most γ sources are suitable for seed irradiation, as long as the size of irradiation space is sufficient and the dose rate allows practical irradiation times. In contrast, fast neutrons are uncharged particles of high kinetic energy and are generated in nuclear reactors or in accelerators. The skilled person should assess the feasibility for seed irradiation with the operators, since not all facilities are suitably equipped and can produce fast neutrons at a low degree of contamination with other radiation.

The two radiation types differ in their physical properties and hence, in their mutagenic activity. γ rays have a lower relative biological effectiveness (RBE) than fast neutrons, which implies that in order to obtain the same biological effect, a higher dose of γ radiation must be given. RBE is mainly a function of the linear energy transfer (LET), which is the transfer of energy along the ionizing track. γ rays produce a few ionizations per micron of path (low LET) and belong to the category of sparsely ionizing radiation. Fast neutrons (high LET, densely ionizing radiation) impart some of their high kinetic energy via collisions, largely with protons within the material.

When radiation passes through tissue, physical events such as ionizations (ejection of electrons from molecules) and excitations (process of raising electrons to a higher energy state) occur and lead to effects in DNA, membranes, lipids, enzymes, etc. Secondly, chemical events are induced that start with the formation of activated molecules, so-called free radicals (OH. and H.) that arise from OH− and H+. If oxygen is present, it reacts readily with radiation-induced free radicals to form peroxyradicals.

In the case of low LET radiation, the formation of peroxyradicals is favoured. In high LET radiation, the formation of hydrogen peroxide (H₂O₂) by recombination of free radicals is favoured. All radicals and hydrogen peroxide can react with biological molecules. Primary damage caused by radiation occurs randomly and is both physiological and genetic. Physiological recovery and repair of DNA are possible to some extent, as non-damaged molecules may take over metabolic processes and DNA repair mechanisms are activated.

Before starting any mutation induction studies, it is most crucial to select suitable doses. For mutation induction, it is advisable to use two to three doses along with a control. The applicable doses will depend on the breeding or research objective, the radiation type and the particular plant material. It is known that plant genera and species and, to a lesser extent, cultivars differ in their radiosensitivity. Radiosensitivity (radiation sensitivity) is a relative measure that gives an indication of the quantity of recognizable effects of the radiation exposure on the irradiated object. The radiosensitivity is influenced by biological factors (such as genetic differences, nuclear and interphase chromosome vol) and by environmental modifying factors (oxygen, water content, post-irradiation storage, and temperature).

Modifying factors greatly affect mutagenic efficiency and reproducibility of results. Oxygen is the major modifying factor, while moisture content, temperature, and storage appear to be secondary, interacting with the oxygen effect. Oxygen shows synergistic action with sparsely ionizing radiation, but oxygen effects during irradiation and post-irradiation storage can easily be prevented by adjustment of seed water content to 12-14% in cereals and most other seeds. In oilseeds such as poppies, the seed water content should be lower, around 7-8%. The critical region is the embryo, but it can be assumed that the water content of the seed and the embryo of most species will be similar. Environmental factors are less important with densely ionizing radiation; thus, for fast neutron radiation, no seed moisture adjustment is necessary.

Unless data on the radiosensitivity of a given plant are already published or known from experience, the mutation induction program should be preceded by a radiosensitivity test. This is done by irradiating the seeds with a range of doses and by growing out the plants under greenhouse conditions. Radiosensitivity is assessed based on criteria such as reduced seedling height, fertility, and survival in the M1 generation. A seedling height reduction of 30-40% is generally assumed to give a high mutation yield. The usefulness of radiation can be judged by mutagenic efficiency, which is the production of desirable changes free from association with undesirable changes. A high dose will increase mutation frequency (the frequency at which a specific kind of mutation or mutant is found in a population of cells or individuals), but will be accompanied by negative features, such as sterility. When selecting the doses, it will be necessary to find a treatment regime providing high mutagenic efficiency.

For fast neutron radiation, dosimetric measurements have to be done during each radiation treatment, e.g., by performing the sulphur threshold detector method, since the neutron flux in the seed irradiation unit is not constant.

The Gray (symbol Gy), the SI (Systéme Internationale) unit used to quantify the absorbed dose of radiation (1 Gy=1 J/kg) replaced the old unit rad; 1 Gy=100 rads or 1 krad=10 Gy. The absorbed dose rate (Gy/s or Gy/min) indicates how much energy the irradiated material absorbs during a given unit of time. The length of exposure and the dose rate determines the radiation dose. Exposure during short times (seconds to a few hours) at a high dose rate is referred to as acute and is most applied in irradiation programs.

Fast neutrons have been shown to be a very effective mutagen. Kornneef et al. (1982) found that about 2500 lines treated with fast neutron at a dose of 60 Gy are required to inactivate a gene once on average (Koornneef, M., Dellaert, L. W. M. and van der Veen, J. H. (1982) EMS- and radiation-induced mutation frequencies at individual loci in Arabidopsis thaliana (L.) Heynh. Mutat. Res.93, 109-123). If the plant genome contains about 25000 genes, it is estimated that about 10 genes are randomly deleted in each line.

FNM offers a number of advantages over using chemical treatment such as EMS. Notably, the treatment is applied to the dried seed, which can be sown at a later date, while with EMS the seed needs either to be sown immediately after treatment, or carefully re-dried for sowing later. However, chemical mutagenesis is particularly useful and methods embodied by the invention are exemplified herein by treatment of seeds with EMS.

After exposing seeds to a mutagen in accordance with a method embodied by the invention, the seeds are typically grown to maturity in controlled conditions and then self-pollinated. The seeds from the mature plant are taken and at least one seed is planted to grow an M2 generation. The M2 generation is screened for alkaloid production. Of course, it is possible to screen the M1 generation, but there are several advantages to screening the M2 generation. Firstly, screening the M2 generation insures that the trait resulting from mutagenesis can be inherited. Secondly, by growing the M2 generation, the basic hardiness of the plant is proven before screening. Thirdly, traits resulting from mutagenesis are generally inherited as recessive genes. Typically the mutated gene will be in the heterozygous state in the M1 generation, and thus the mutation will be masked by the dominant (non-mutated) form of the gene. In the M2 generation, however, in a proportion of the plants the gene will be in the homozygous state, and the effect of the mutation apparent.

The M2 plants can be grown to produce an immature capsule, but it is possible to save time and labor if the plants are screened at an earlier stage of growth. It is recommended that the plants be screened at a point beginning at the 6 leaf stage, up to the 10 leaf stage. Screening at this early stage advantageously allows many plants to be managed in a small space.

The screening process itself is the most labor intensive and as such, to improve return on labor, generally only plants that appear healthy have conventionally been screened. However, the present inventors contrary to conventional practice, also screened plants which had substantially lighter colour leaves than the parent plant and other plant lines generated as described above. As the lighter colour could indicate a serious defect such as a nutrient deficiency or a reduction in chlorophyll content in their leaves, these plants would have been expected to have reduced vigour, which would adversely impact on commercially important characteristics of the plants such as one or more of rate of maturation, capsule number, poppy straw and/or latex yield and thereby, overall codeine yield. Nevertheless and contrary to expectations, the present inventors found that such plants could have high codeine content and increased amount of codeine relative to thebaine compared to the parent plant utilised, and so be useful as a commercial field crop.

In the screening process, the objective is to measure each plant for codeine content relative to morphine, and thebaine content. Additional confounding alkaloids such as oripavine can also be measured.

This can be accomplished by extracting, for example, poppy straw into a liquid buffer or by dissolving a latex sample into a buffer. The buffer solutions are placed onto 96 well trays and fed mechanically through any of the high-throughput HPLCs available on the market. In a preferred embodiment, latex can be very rapidly screened utilizing isocratic ultra-high performance liquid chromatography (UPLC).

A very rapid and efficient screening method is desirable to test sufficient plants for finding an advantageous mutation. Suitable alkaloid screening methods are for instance described in WO 2009/143574 and WO 2009/109012. Furthermore, by using UPLC apparatus with a very sensitive UV detector (e.g., a Waters Acquity UPLC) it is possible to quantify very low levels of alkaloid, meaning that even very small plants can be tested. Additionally, very rapid screening (e.g., 0.8 minute) of each plant can allow over 1000 samples to be analysed daily. As a result, the entire screening process may be conducted quickly.

Plants identified by the screening process to have an altered alkaloid profile of interest are grown further and examined in more detail. According to the procedure herein, a second sample is taken from about 3% of plants to clarify or confirm the results of the initial screen. A more precise gradient UPLC method can then be used to obtain more accurate peak identification and quantification. Plants confirmed to have the desired alkaloid profile are transplanted to 200 mm (approx. 8 inch) pots for growing to maturity.

As used herein, the term “poppy straw” or “straw” is to be taken to mean the straw material obtained when the mature poppy capsules of a Papaver somniferum plant are collected, and threshed to remove the seeds to form a straw.

As used herein, the term “opium” is to be taken to mean the air-dried, milky exudation (i.e., the latex) from incised, unripe poppy capsules of a Papaver somniferum plant.

As used herein, the term “concentrate of poppy straw” or “CPS” is to be taken to mean the material arising when poppy straw has entered into a process for the concentration of its alkaloids in either liquid, solid or powder form which contains the phenanthrene alkaloids of the opium poppy.

As used herein, the phrase “stand of Papaver somniferum ” or “stand of stably reproducing Papaver somniferum ” or the like, refers to a group of two or more Papaver somniferum plants or stably reproducing Papaver somniferum plants located together. Typically, the stand will comprise at least 3, 4, 5, 6, 7, 8, 9, 10, 11, 12, 13, 14, 15, 16, 17, 18, 19, or 20 or more Papaver somniferum plants located together e.g., 30, 40, 50, 60, 70, 80, 90 or 100 or more of the plants. Typically, the plants are grown or growing in a field exposed to ambient environmental conditions.

As used herein, the term “alkaloid combination” is to be taken to refer to a combination of alkaloids wherein the alkaloids comprise morphine, codeine and thebaine. The alkaloid combination can also include and oripavine in the context of plant embodied by the invention or a parent plant as described herein. In further embodiments of the present invention, the alkaloid combination may comprise one or more additional alkaloids as may be selected from the group consisting of, codeinone, neopinone, protopine, laudanine, laudanosine, salutaridine, reticuline, papaverine and noscapine, in addition to morphine, codeine, thebaine and when included, oripavine.

A “stably reproducing” Papaver somniferum poppy plant as described herein refers to a poppy plant that is stably reproducing as required to plant and harvest seed from poppy crops over multiple generations where each generation would be suitable, without seed selection, for commercial planting of a field crop or stand of plants exhibiting the desired alkaloid characteristic(s). Further, a stably reproducing poppy plant in accordance with the invention has the desired alkaloid characteristics as described herein, and when self-pollinated, or cross pollinated by a plant with the same genes controlling alkaloid content, produces a subsequent generation of plants which all have the same genetic potential to substantially have the same desired alkaloid characteristics as the parent plant. Moreover, in the absence of pollination with pollen from other chemotypes (e.g., conventional morphine accumulating plants), the line will continue to produce similar plants over multiple generations, without the need for selection to maintain the desired alkaloid characteristic.

As above, the term “stably heritable” as used herein is to be taken to mean the Papaver somniferum produces a subsequent generation of plants which all have the same genetic potential to substantially have the same alkaloid characteristics as the parent plant, when the plant is self-pollinated, or cross pollinated by a plant with the same genes controlling alkaloid content as described above. As above, in the absence of pollination with pollen from other chemotypes (e.g., conventional morphine accumulating plants), the line will continue to produce similar plants over multiple generations, without the need for selection to maintain the specified alkaloid characteristic.

As used herein the term “trait” is to be taken to mean a distinct heritable phenotypic characteristic. The desired trait(s), once established are consistently inherited by substantially all the progeny. To maintain the desired traits, care should be taken to prevent cross-pollination with normal plants unless such cross-pollination is part of a controlled breeding program.

Examples of desired trait(s) and/or alkaloid characteristics of Papaver somniferum plants embodied by the invention which can be passed on to future generations (e.g., progeny and descendent plants thereof) include a) a high codeine content in poppy straw, latex and/or opium, b) a high codeine content relative to one or more confounding alkaloids in the production of codeine, particularly at least thebaine in poppy straw, latex and/or opium, and c) a lighter leaf colour as described herein in combination with a) or b).Typically, a plant embodied by the invention has a trait for lighter leaf colour as described herein that is controlled by a single recessive gene, wherein the trait is associated with at least one of an increase in codeine content and a reduction in thebaine content relative to codeine content, in the poppy straw or opium of the plant.As used herein, the term “substantially no” when referring to thebaine content means that thebaine constitutes less than 10% by weight, preferably less than 5.0% by weight, more preferably less than 2% by weight, and most preferably, less than 1% of the alkaloid combination of morphine, codeine and thebaine, and most preferably the alkaloid combination of morphine, codeine, thebaine and oripavine, of the poppy straw, concentrate of poppy straw or opium.

The term “substantially no”, when referring to morphine, oripavine and alkaloids other than thebaine as used herein, means that each specified alkaloid respectively constitutes less than 1% by weight, preferably, less than 0.5% by weight, more preferably, less than 0.3% by weight, and most preferably, from 0% to 0.2% by weight of the alkaloid combination of morphine, codeine and thebaine, and most preferably, the alkaloid combination of morphine, codeine, thebaine and oripavine, of the poppy straw, concentrate of poppy straw, latex or opium.

As used herein, the “M1 population” is the seeds and resulting plants exposed to a mutagenic agent, while “M2 population” is the progeny of self-pollinated M1 plants, “M3 population” is the progeny of self-pollinated M2 plants, “M4 population” is the the progeny of self-pollinated M3 plants, and generally “Mn population” is the progeny of self-pollinated Mn-1 plants.

The desired trait(s) can be transferred into Papaver somniferum lines having other characteristics (e.g., different height, early or late maturity, or having disease resistance) by cross pollinating a plant embodied by the invention with the second parent plant, collecting F1 seed, growing a F1 plant which is allowed to self-pollinate and collecting the F2 seed. The F2 seed may then be grown, and individual plants that have the desired leaf colour, codeine and/or other alkaloid phenotypic characteristic(s) as described herein may be selected according to the methods herein, along with the other desired characteristic(s) e.g., disease resistance. Further selection can then be undertaken if desired in the F3 and/or subsequent generations in order to produce highly uniform lines. Alternatively, a high codeine plant embodied by the invention can be used as the first parent in a crossing program. A skilled operator will be able to apply variations to this method as well known in conventional plant breeding.

Conducting test crosses with plants of known genotype can provide information regarding the genetic changes introduced through mutation. The characteristics of the F1 generation produced by crossing to a normal parent will indicate whether a trait inherits as a recessive or dominant gene. Self-pollinating the F1 plants and determining the phenotypes of the subsequent F2 population of plants will provide information regarding the numbers of genes responsible for particular characteristics.

As used herein, the term “descendent” plant is meant a Papaver somniferum plant which is the progeny of a plant embodied by the invention or is derived from a plant embodied by the invention such as a granddaughter plant, great grandaughter plant and the like, or a plant as may be obtained by cross-pollinating a plant embodied by the invention (or e.g., a progeny plant thereof) with another Papaver somniferum poppy line having desirable trait(s) of interest, testing the progeny at the F2 or F3 or subsequent generations, and selecting progeny exhibiting the desired trait(s) through incorporation of gene(s) from the new parent plant. In preferred embodiments of the invention, seed from Papaver somniferum which upon harvesting of their capsules produce a poppy straw containing codeine as the sole or predominant alkaloid in the alkaloid combination of morphine, codeine, thebaine and oripavine, or alternatively, which upon the drying of latex from their immature poppy capsules will yield an opium containing codeine as the sole or predominant alkaloid of the alkaloid combination, may be used to provide a plant embodied by the present invention. Typically, the poppy straw of the Papaver somniferum parent plant will contain codeine constituting about 40% by weight or greater of the alkaloid combination. Preferably, codeine will constitute about 45%, 50%, 55%, 60%, 65%, 70%, 75%, 80%, 85%, or 90% by weight or greater of the alkaloid combination, e.g., 91%, 92%, 93%, 94%, 95%, 96%, 97% or even 98% or greater.

Typically, the parent plant yields a poppy straw having a codeine content above 0.8% w/w of the straw. Preferably, the codeine content of the straw is about 1.0%, 1.5%, or 2.0% w/w or greater and generally will be in a range of from about 2.0% to about 4.2% w/w of the straw or greater, e.g., 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, or 4.2%, or even greater (e.g., up to 4.5% or 5% or more). Typically, the codeine content of the poppy straw will be at least about 2.5% w/w of the straw and more generally, in a range from about 2.5% to about 4.19% w/w.

Typically, the morphine content of the poppy straw of the parent plant is about 0.05% w/w or less of the poppy straw, e.g., 0.045%, 0.04%, 0.035%, 0.03%, 0.025%, 0.02%, 0.015%, or less 0.01%.

Typically, the thebaine content of the poppy straw of the parent plant is about 3% w/w of the straw or less, e.g., 2.5%, 2%, 1.5%, 1%, 0.9%, 0.8%. 0.7%, 0.6%, 0.5% or 0.4% w/w or less. Most preferably, thebaine constitutes from about 0.4% to about 0.6% w/w of the poppy straw.

In at least some embodiments, in the poppy straw of the Papaver somniferum parent plant the ratio of codeine and thebaine to the alkaloid combination of morphine, codeine, thebaine and oripavine on a w/w basis, is between about 0.90 and 1.00, e.g., in a range from about 0.91 and 1.00, about 0.92 and 1.00, 0.93 and 100, 0.94 and 1.00, 0.95 and 1.00, 0.96 and 1.00, 0.97 and 100, 0.98 and 1.00, and about 0.99 and 1.00. Typically, the ratio is between about 0.98 and 1.00 and even more preferably, about 0.99 and 100.

Typically, the oripavine content of the parent plant straw is less than the thebaine content of the straw. Most typically, the oripavine content of the straw is about 1% w/w of the poppy straw or less and more preferably, is about 0.9%, 0.8%, 0.7%, 0.6%, 0.5% w/w or less. Most preferably, the oripavine content of the poppy straw is about 0.4%, 0.3%, 0.2%, or 0.1% w/w of the straw or less and typically, there is no oripavine in the straw.

In preferred embodiments, the straw of the parent Papaver somniferum plant(s) contains substantially no morphine and/or substantially no oripavine in the alkaloid combination of morphine, codeine, thebaine and oripavine.

In particularly preferred embodiments, the straw of the parent Papaver somniferum plant(s) contains substantially no thebaine in the alkaloid combination of morphine, codeine, thebaine and oripavine. Most preferably, there is substantially no morphine, thebaine and oripavine in the alkaloid combination of morphine, codeine, thebaine and oripavine.

Stably reproducing Papaver somniferum parent plants suitable for use in providing a plant having a stably heritable codeine chemotype characterized by higher codeine content by weight than a combined morphine and thebaine content in accordance with a method of the present invention are, for example, described in International Patent Publication No. WO 2009/143574 in the name of Tasmanian Alkaloids Pty Ltd, the entire contents of which is incorporated herein in its entirety by cross-reference. Those plants were obtained by crossing plants of Papaver somniferum which produced thebaine as the sole or predominant alkaloid in the alkaloid combination of morphine, codeine, thebaine and oripavine in poppy straw or latex from their immature poppy capsules, with Papaver somniferum plants having a morphine chemotype that is, plants in which morphine is the predominant alkaloid in poppy straw from the plants or latex obtained from their immature poppy capsules. More particularly, plants of Papaver somniferum were obtained having codeine as the predominant alkaloid and substantially no oripavine or morphine by the cross. Plants were also obtained having codeine as the predominant alkaloid and substantially no oripavine, morphine, thebaine or any other alkaloids.

The high thebaine plants used in the cross described immediately above are described in International Patent Publication No. WO 2009/109012, the entire content of which is also incorporated herein in its entirety by cross-reference. The high thebaine chemotype of those plants is believed to be the result of two independent genetic changes in the plants; one genetic change controlling the accumulation of thebaine and oripavine compared with morphine and codeine, and the second genetic change controlling the accumulation of thebaine compared with oripavine. More particularly, the two independent genetic changes were provided by mutations in two genes, the first gene blocking thebaine from being converted to neopinone, and oripavine from being converted to morphinone (as exemplified by the TOP 1 mutation described below), and the second gene blocking a pathway between thebaine and oripavine, see the metabolic pathway set out in Scheme 1 below.

More particularly, Papaver somniferum is postulated to have two biosynthetic pathways from thebaine to morphine as shown in Scheme 1. Pathway A via neopinone, codeinone and codeine was proposed by Parker, H. I., J. Am. Chem. Soc., 94, 1276-1282 (1972). Pathway B via oripavine and morphinone was proposed by Brochmann-Hanssen, E., Planta Med., 50, 343-345 (1984). The enzyme codeinone reductase (NADPH) is believed to be active in both pathways, reducing codeinone to codeine and morphinone to morphine. Further, the TOP1 mutation (Millgate et al., Morphine-pathway block in topl poppies. Nature, Vol. 431, 413-414, 2004) affects both pathways, preventing thebaine being converted to neopinone in Pathway A, and preventing oripavine being converted to morphinone in Pathway B. In particular, the TOP1 mutation appears to block demethylation of the enol ether which converts thebaine to neopinone, as well as the demethylation of the same enol ether in oripavine.

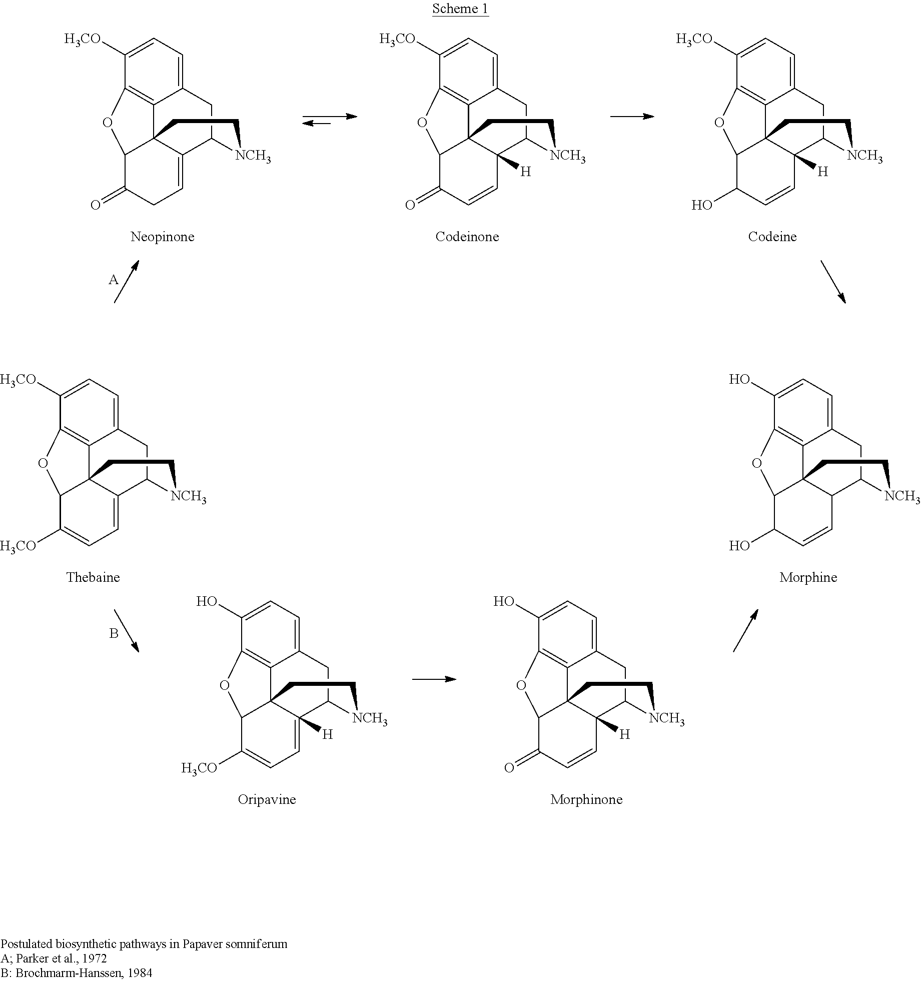

Thus, when the gene blocking the conversion of thebaine to oripavine whereby the production or activity of the phenolic 0-demethylation enzyme that converts thebaine to oripavine is substantially inhibited and described in WO 2009/109012 as producing the high thebaine poppies in conjunction with the exemplified TOP1 mutation (also known as the Norman mutation)) is introduced into plants lacking that mutation (i.e., into Papaver somniferum plants with a morphine chemotype as described in WO 2009/143574), plants with a codeine phenotype are produced, and these plants can be utilized in methods described herein to produce plants in accordance with the present invention. Typically, a plant produced in accordance with a method embodied by the invention is a plant which upon the harvesting of its poppy capsules will yield a poppy straw having a higher absolute codeine content and a lower thebaine to codeine content, than the straw of the parent plant from which the plant of the invention is derived.

Typically, the poppy straw of a plant produced in accordance with a method of the invention will have a codeine content which is about 4% or greater than the poppy straw of the parent plant on a w/w basis, e.g., about 10% w/w or greater, or even about 15% w/w or greater. Thus, the straw of plant embodied by the invention may have about 4%, 5%, 6%, 7%, 8%, 9%, 10%, 11%, 12%, 13%, 14% or even 15% w/w or more codeine than the poppy straw of the parent plant.

Typically, the poppy straw of a plant produced in accordance with a method of the invention will have a thebaine content which is about 10% or less than the poppy straw of the parent plant on a w/w basis. For example, the poppy straw of the plant may have a thebaine content which is about 15%, 20%, 25%, 30% or even 40% less on a w/w basis compared to the straw of the parent plant.

Typically, the poppy straw of a plant embodied by the invention will have a thebaine to codeine ratio of about 25%, 24%, 23%, 22%, 21%, 20%, 19%, 18%, 17%, 16%, 15%, 14%, 13%, 12%, 11%, or 10% or less on a w/w basis, more preferably about 9.5%, 9.0%, 8.5%, 8.0%, or 7.5% or less on a w/w basis and most preferably, about 7.0% w/w or less, 6.5% w/w or less, about 6% w/w or less, about 5.5% w/w or less, or even about 5% w/w or less.

Typically, the poppy straw of a plant embodied by the invention will have a codeine content that is, is about, or is greater than about, 0.8%, 0.832%, 1.0%. 1.5%, or 2.0% w/w of the poppy straw or greater. Generally, the codeine content of the poppy straw will be in a range of at least about 2.0% to about 5% or greater on a w/w basis of the poppy straw (e.g., about 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, or even 4.2%, 4.3%, 4.4%, 4.5%, 5%, or 6% w/w or greater of the poppy straw).

The term “% w/w” or “% w/w basis” or the like as used herein in the context of the content of a specified alkaloid relative to poppy straw is meant the content of the alkaloid in poppy straw obtained from mature, field dried Papaver somniferum.

Plants contain a variety of pigments that contribute to both prominent visual features (e.g., flower colour) and important physiological processes. One of the most well-known classes of plant pigments are the chlorophylls (e.g., chlorophyll a and b). These pigments play essential roles in photosynthesis including the capture and harvesting of light energy from the sun. Humans recognize pigment colour by perceiving the visible light (i.e., wavelengths between ˜390 to ˜700 nm) which is reflected or transmitted by the pigment. For example, the characteristic green colour of chlorophylls can be explained by the fact that chlorophylls absorb light in the violet-to-blue and red light regions, leaving a considerably wide gap in the absorption spectrum known as the ‘green window’ (Chen, M. (2014) Chlorophyll modifications and their spectral extension in oxygenic photosynthesis. Annual Review of Biochemistry, 83, 317-340). The reflectance of visible light in this so-called green window give chlorophylls their green colour.

The importance and prevalence of chlorophylls explains the abundance of green-coloured tissues in plants. However, many other non-green plant pigments also occur and similarly provide vital physiological roles. A group of pigments called carotenoids confer yellow-to-red coloration to flowers and fruits. Along with chlorophylls, carotenoid pigments constitute an essential component of the photosystem light-harvesting complexes involved in photosynthesis (Tanaka, Y., Sasaki, N. and Ohmiya, A. (2008) Biosynthesis of plant pigments: anthocyanins, betalains and carotenoids. The Plant Journal, 54, 733-749). Carotenes are a class of the carotenoid pigment family and include β-carotene; a major carotenoid pigment in higher plants (Hopkins, W. G. and Hüner (2004) Introduction to Plant Physiology (3^{rd} ed.) Wiley and Sons (MA, USA)). Whilst carotenoid pigments including β-carotene can serve as accessory pigments in the capture of light energy, their principal function is that of an anti-oxidant, preventing photooxidative damage to the chlorophyll molecules within the chloroplast (Raven, P. H., Evert, R. F. and Eichhorn, S. E. (1999) Biology of Plants (6^{th} ed.) W. H. Freeman and Company (NY, USA)). Further, the carotenoid pigments violaxanthin, antheraxanthin and zeaxanthin, also present in chloroplasts, function in a process known as the xanthophyll cycle which serves to dissipate excess energy and thereby provide photoprotection (Hopkins, W. G. and Hüner (2004), vide supra). Although present in leaf tissues, the colours of carotenoid pigments are generally masked by the more abundant chlorophylls. However, carotenoids and other plant pigments may become visible in plant leaves under certain conditions. For example, during autumn chlorophyll pigments are degraded in leaves of deciduous plant species. During this leaf senescence process the more stable carotenoid pigments are revealed, resulting in the characteristic orange and yellow foliage colours of autumn (Hopkins and Hüner 2004, vide supra). Alternatively, other species may produce brilliant red foliage during autumn (e.g., Quercus rubra; red oak) due to the accumulation of anthocyanin pigments in their leaves (Lee, D. W. and Gould, K.S. (2002) Why leaves turn red. American Scientist, 90, 524-531).

Anthocyanins belong to another major class of plant pigments called flavonoids.

These phenylpropanoid secondary metabolites have a wide colour range, ranging from pale-yellow to blue. Notably, the anthocyanins are responsible for the orange to blue colours found in many flowers, leaves, fruits, seeds and other tissues (Tanaka et al. 2008, vide supra). Other flavonoids, such as the flavonols, are commonly found in leaves and along with flavones are very pale-yellow. Whilst these pigments can be mostly invisible to the human eye, their ultra-violet (UV) absorbing properties provide colour and patterns that serve to attract insect pollinators in addition to protecting against UV damage (Winkel-Shirley, B. (2002) Biosynthesis of flavonoids and effects of stress. Current Opinion in Plant Biology, 5, 218-223; Tanaka et al. 2008, vida supra).

As described herein, in at least some embodiments of the invention, plants having a noticeably lighter leaf colour than the parent plant from which they were derived were found to nevertheless have an increased codeine content than the parent plant, the colour ranging from a visually lighter green to yellow coloured leaves. To evaluate the colour of the leaves, spectroscopic measurements were taken to obtain three-dimensional (3D) colour coordinates. Chromaticity coordinates were then calculated from the spectrophotometer results in order to reduce the dimensionality of the data and to obtain dominant wavelength values for each plant line evaluated. The dominant wavelength, as measured in nanometers (nm), is a measure of the hue of an object's colour and is used herein to describe leaf colour. Methods of colour measurement and colour description as described herein are well known in the art and are described in colourimetry texts including, for example, Wyszecki, G., & Stiles, W. S. (1982), Color Science: concepts and methods, quantitative data and formulae (2nd Ed.; New York: Wiley).

The dominant wavelength values of the measured leaves of plants described herein corresponded to the green-yellow spectral wavelength region. Thus, the respective plants can be described as all having green-yellow leaves, and plants embodied by the invention having lighter coloured leaves than the parent plant can be described as having e.g., light green-yellow leaves or for instance, very light green-yellow leaves, compared to the parent plant.

Hence, the term “green-yellow” as used herein in the context of the leaf colour of a plant embodied by the invention and/or parent plant, is to be taken to mean green-yellow in the context of the green-yellow colour spectrum.

Plants embodied by the invention which are grown in the field (and so are exposed to ambient weather conditions during their development) typically have leaves that are markedly lighter in colour than if those plants were grown in a planthouse. As such, plants of the invention that are field grown will typically have a dominant wavelength value that is greater than that if the plants were planthouse grown plants.

The term “planthouse” is used herein herein to refer to either a plastic covered “polyhouse” or to a greenhouse.

Typically, a plant of Papaver somniferum having a lighter leaf colour embodied by the invention as described above will have green-yellow leaves exhibiting a dominant wavelength value in a range of from about 561 nm to 568 nm or greater, but not exceeding 570 nm. In other embodiments, plants embodied by the invention, or identifiable by the invention, predominantly have leaves exhibiting a dominant wavelength that is different to the dominant wavelength of the leaves of the parent plant. Typically, the dominant wavelength of at least a majority of the leaves of a plant of the invention is different to the dominant wavelength of at least the majority of the leaves of the parent plant.

Most typically, the dominant wavelength value of a plant embodied by the invention will be in a range of from about 561 nm to about 568 nm e.g., a dominant wavelength of about 562 nm, 563 nm, 564 nm, 565 nm, 566 nm, 567 nm, or 568 nm. It will also be understood that all ranges with the dominant wavelength identified above are expressly encompassed. For instance, a plant embodied by the invention having a lighter leaf colour may have a green-yellow leaf colour exhibiting a dominant wavelength in a range of from about 561 nm to about 570 nm, from about 561 nm to about 569 nm, from about 561 nm to about 568 nm, from about 562 nm to about 568 nm, from 562 nm to about 567 nm, or from 563 nm to about or 566 nm. A method for the measurement of the dominant wavelength is exemplified further below.

Typically, the dominant wavelength is determined by reflective spectrophotometry on the adaxial surface of healthy, leaves using D65 illumination, wherein healthy leaves are characterised as leaves being free from visible signs of disease, senescence, nutrient deficiency/toxicity, and other forms of stress (e.g., temperature-, water, or herbivory-related stress).

The leaf colour can be evaluated at any stage up until maturity of a plant embodied by the invention and compared with the leaf colour of the parent plant at the same stage of development, such as running up, late running up, bud in apex, early hook, hook, mid-hook, upright bud and first flower stages. In particularly preferred embodiments, leaf colour is assessed in the early hook or hook stages and more preferably in the early to mid-hook growth stages. As the leaf colour difference in light-leaf colour plants embodied by the invention is more pronounced in field grown plants compared to field grown parent plants, it is desirable that the evaluation of leaf colour is undertaken on field grown plants. Likewise, it is preferable that leaf colour comparisons be made between plants grown in the same location and/or under the same conditions. The determination of conditions for growing plants for purposes of leaf colour comparisons are well within the expertise of a person of ordinary skill in the art. Conditions suitable for growing plants in a planthouse for the purpose of leaf colour comparisons are, for instance, further described below.

Whilst dominant wavelength is exemplified herein as a measure of the leaf colour, other parameters may be used to evaluate leaf colour of a plant embodied by the invention relative to a parent plant as described herein. For example, other methods for measuring leaf colour may include evaluating the content of one or more pigments in leaves responsible for the leaf colour a plant, such as cholorophyll, (e.g., cholorophyll a and/or chlorophyll b), accessory pigments such as one or more carotenoid(s), anthocyanins, and mixtures of the foregoing, and all suitable alternative methods are expressly encompassed.

Typically, a plant embodied by the invention has a reduced leaf pigment content comprising a reduced level of at least one of chlorophylls and carotenoids in the leaves of the plant.

Most typically, the reduced level of chlorophylls comprises a reduced level of both chlorophyll a and chlorophyll b. In at least some embodiments, the level of chlorophylls may be reduced by least 10% by weight. In some embodiments, the level of chlorophylls may be reduced by up to 20% by weight or even more (e.g., by 10%, 11%, 12%, 13%, 14%, 15%, 16%, 17%, 18%, 19%, 20% or even by 25%, 30%, 35%, 40%, 45%, or 50% by weight or more).

The reduced level of carotenoids will typically comprise a reduced level of at least one of lutein and β-carotenoid.

Despite having a lighter leaf colour on the adaxial surface of leaves wherein the lighter leaf colour is associated with a modified alkaloid profile in poppy straw or opium, the plants in accordance with the invention as described herein are nevertheless healthy, viable plants exhibiting good vigour with no symptoms of disease or nutrient deficiency.

As used herein, the term “field grown” is to be taken to mean plants grown in situ from seed sown in the field and plants that are grown to maturity in the field from transplanted seedlings raised from seed e.g., in a planthouse.

In at least some embodiments, a plant of the present invention may be asexually reproduced, including via methods such as tissue culture.

Recovering the codeine from dried poppy straw, opium or concentrate of poppy straw is a process well established in the art. Until now, thebaine has been extracted from this plant species either as a part of the process of extracting morphine and codeine, or more recently as part of the process of extracting thebaine and oripavine.

For example, the poppy straw can be treated with a small amount of lime and water to soften the capsules and to form a free base of the alkaloids. Countercurrent extraction of the softened straw with methanol, ethanol or other suitable solvent forms a solvent/water extract or “miscella” containing the alkaloids, with morphine at a concentration of about 1 g/L where the straw is from standard Papaver somniferum. The volume of the miscella is reduced about 30 x under vacuum to produce an aqueous concentrate. Any thebaine can be extracted from the aqueous concentrate using one or more liquid/liquid partitioning extraction steps using suitable solvent(s) (e.g., toluene or xylene), adjusting pH for the best separation of thebaine. Codeine remains in the aqueous phase and Codeine CPS can be precipitated from the aqueous phase by pH adjustment.

An alternative means of producing alkaloids is to grow plant cells or plant organs such as shoots or roots in culture. Cell culture or organ culture are means of producing alkaloids without being subject to the vagaries of climate and other uncertainties associated with crop production. The general methods of establishing cell cultures, root cultures and shoot cultures for the purpose of alkaloid production are provided by M. F. Roberts, Production of alkaloids in plant cell culture. In Alkaloids, Biochemistry, Ecology, and Medicinal Applications, Edited by Roberts and Wink, Plenum Press, New York 1998, pages 159-197, the contents of which is hereby incorporated by reference in its entirety. The first step in producing cell cultures is to establish growth of callus. One way of achieving this for Papaver somniferum is provided by Chitty et al.(2003)., Genetic transformation in commercial Tasmanian cultivars of opium poppy, Papaver somniferum L., and movement of transgenic pollen in the field. Functional Plant Biology 30: 1045-1058. In this method, seeds are surface sterilized by washing for 30-60 seconds in 70% ethanol, then in 1% (w/v) sodium hypochlorite solution plus 1-2 drops of autoclaved Tween 20 for 20 minutes with agitation. Seeds are then rinsed three to four times in sterile distilled water, or until no smell of bleach remains, and placed on B50 agar medium (Gamborg et al. 1968 Nutrient requirements of suspension cultures of soybean root cells. Experimental Cell Research 50, 151-158). Dishes are sealed with parafilm and imbibed at 4° C. for 24 to 48 hours. Seeds are germinated at 24° C. in a 16 hour light-8 hour dark cycle. Hypocotyls are excised from seedlings after 7-8 days of culture, cut into 3-6 mm pieces (usually 1-3 explants per seedling) and placed onto callusing media. Culture media consists of B50 macronutrients, micronutrients, iron salts and vitamins (Gamborg et al. 1968) and sucrose at 20 g/L. pH can be adjusted with 1M KOH to pH 5.6 and 0.8% Sigma agar (A1296) can be used as a gelling agent.

All media should be autoclaved at 121° C. for 20 minutes. B50 medium contains no growth regulators and is used to germinate seeds aseptically, maintain embryogenic callus, and regenerate shoots and plantlets. Callusing Medium (CM) is B50 medium plus 2,4-dichlorophenoxy acetic acid (2,4-D) at 1 mg/l, added prior to the medium being autoclaved.

To generate a cell suspension culture (method from Staba et al. 1982, Alkaloid production from Papaver tissue cultures, Journal of Natural Products, 43,256-262), callus cultures can be transferred into 125 mL Erlenmeyer flasks containing 25 mL of liquid RT medium (Khanna and Khanna 1976, Ind J Exp Biol 14,628) supplemented with either 5 ppm BA for the growth of shoots or 0.1 ppm 2,4-D for the development of cell suspensions. Cultures can be grown at 28° C. on an orbital shaker (78 rpm) with 15 hours of light per day. In particular, cell cultures can be grown as a batch culture where the cells multiply in a liquid medium which is being continuously agitated to maintain the cells as small aggregates and to maintain oxygen levels. Typically, after the initial inoculation there is a lag phase, followed by an exponential growth phase, which is then followed by a stationary phase where the growth becomes limited by lack of some components of the medium. Often, secondary plant products such as alkaloids are accumulated while the culture is in the stationary phase. For some products, alkaloid production can be induced by adding elicitors such as fungal cell extracts. There are also systems of continuous or semi-continuous culture where fresh medium is added either continuously or semi-continuously while cells or media are likewise removed for alkaloid recovery. Critical to the success of any cell culture system is the establishment of high yielding cell lines. Generally, selection is required to select individual plants, or individual cell cultures that produce the required alkaloid. For the production of codeine, a rapid HPLC or UPLC method such as those described in this application could be modified to test cell lines for codeine production.

Techniques such as root culture including hairy root culture where roots are transformed with Agrobacterium rhizogenes may also be a viable means of producing codeine in culture. A method for transformation of Papaver somniferum cultures with A. rhizogenes is, for instance, described in Yoshimatsu and Shimomura (1992), Transformation of opium poppy (Papaver somniferum L.) with Agrobacterium rhizogenes MAFF 03-01724. Plant Cell Reports 11,132-136. A person skilled in the art of cell and organ culture would also be able to envisage other means of growing plant cells derived from plants embodied by the present invention in order to produce codeine.

Methods for sampling leaf latex, and measuring the content of codeine, thebaine, morphine, oripavine and other alkaloids as described herein in poppy straw, latex, opium and concentrate or of poppy straw are well known to the skilled person, see for instance WO 2009/143574 and WO 2009/109012.

The invention is further described below with reference to a number of Examples. The Examples are not intended and should not be construed as limiting the invention in any way.

### EXAMPLE 1

### EMS Mutagenesis treatment of Seed of a Papaver Somniferum Line Commercially Grown for the Production of Codeine

The seed of a stably reproducing Papaver somniferum “Tasman” parent line (PW08-2308) which produces codeine as the predominant alkaloid and relatively low levels of thebaine and essentially no morphine or oripavine as described in WO 2009/143574 was subjected to EMS mutagenesis treatment.

In brief, 2×2 g lots of seed from the commercially grown Papaver somniferum “Tasman” line (PW08-2308) were utilised in the present study. Each 2 g seed lot was placed in a 15 cm×15 cm square of porous mesh curtain material and the corners of the material were tied together to form a pouch. The bags of Tasman seed were placed in separate 250 mL flasks filled with chilled (4° C.) 100 mM phosphate buffer (pH 7). The flasks were sealed with a rubber stopper and placed in a refrigerator at 4° C.) where they were left overnight.

The next the day the bags of seed were removed from the phosphate buffer solution, and a new solution of 0.7% EMS (ethyl methanesulfonate) in phosphate buffer was prepared by adding 1.75 mL of EMS to 250 mL phosphate buffer. The bags of seed were added to the EMS solution, the flask was capped and the solution stirred on a magnetic stirrer (without heat). Seed was treated in this way for 5 hours.

At the end of each treatment period the bags of seed were removed from the

EMS solution and rinsed under running water for 30 minutes. The seed was then left overnight in 200 mL distilled H₂O at 4° C. The next day seeds were again rinsed under running water for 30 minutes, spread thinly on a layer of tissue paper and left to air-dry for 2 hours to facilitate ease of handling when sowing.

The air-dried mutagenized M1 seed was immediately sown in 14 cm diameter pots filled with potting soil (a 50:50 mix of coarse and composted pine bark, with Osmocote™ slow release fertiliser added). Seeds were sown at the rate of about 10 per pot and 96 pots of each line/EMS treatment combination were sown. After sowing the seed was covered with a fine layer of vermiculite. Watering was via overhead sprinklers for the first 14 days followed by drip irrigation through to maturity. Plants were fertilised by weekly application of a liquid fertiliser. The M1 generation was grown in an enclosed (plastic covered) planthouse under natural day lengths in Westbury, Tasmania, Australia. Individual M1 plants were self-pollinated through the use of a paper bag placed and secured over individual flower buds prior to anthesis.

At maturity, M2 seed was harvested from each M1 plant and placed in a separate labelled seed packet. An equal amount (0.08 g) of M2 seed was then taken from each packet to produce four bulk M2 seed samples.

Bulked M2 seed was sown in a field breeding nursery (Weetah, Tasmania, Australia) on 22 Sep. 2011. The seed was sown in 5 m long×1.6 wide plots, with 6 rows per plot, using a custom-built-trial-seed drill. Seventy plots of the Tasman/5 hr treatments were sown, with approximately 0.24 g of seed used to sow each plot. Basal fertilizer was incorporated into the soil at the time of sowing.

Plants were inspected regularly throughout development to screen for interesting and potentially useful phenotypes. At early stages of development plants with phenotypes of interest were marked with a flag to ensure they were examined further. After ‘running up’ all of these plants were tagged with a label that described their phenotype. All labelled phenotypic mutants were self-pollinated by placing and securing a paper bag over the primary flower bud prior to anthesis. In addition, 1200 ‘Tasman’ M2 plants, with normal phenotypes, were selected at random and self-pollinated by securing a paper bag over the primary flower bud prior to anthesis.

M3 seed from each of the M2 plants was harvested separately when plants were fully mature and dry (February 2012) and were assigned line numbers starting with EM3 or EM4.

M3 seed from ‘Tasman’ phenotypic mutant lines was grown in the planthouse during April 2012 to further examine their phenotype and bulk M4 seed for future field trials. Plants were grown 6-per-pot in 14 cm diameter pots in commercial potting soil, under a 16hr photoperiod.

### EXAMPLE 2

### M3 Field Trials

1. M3 Alkaloid/Phenotype Screening Trial

A number of M2 mutant plants with interesting phenoytpes were identified in the M2 populations grown (Weetah, Tasmania, Australia) during the 2011/12 season as outlined in Example 1. Some of the phenotypes observed in the M2 field grown plants were not evident when the M3 generation was grown in the planthouse, and these M3 lines were excluded from further investigations. Overall, 982 M3 lines that appeared worthy of further investigation were advanced to field trials (Hagley, Tasmania, Australia) during the 2012/13 poppy growing season.

M3 ‘Tasman’ mutant lines produced in Example 1 were sown in one or more of three different field trials, to assess, various aspects of their alkaloid content, alkaloid profiles and/or phenotypes.

The first of these trials, the M3 alkaloid screening trial, was conducted in a paddock (Hagley, Tasmania, Australia) during the 2012/13 poppy growing season. Each M3 line was sown in a single, 5 m long row within a 5 m×1.8 m plot that contained six such rows. Multiple replicates of the ‘Tasman’ parent line were grown throughout the trial. A visual assessment of plant phenotype was conducted prior to flowering. Plants were allowed to mature and dry under field conditions, and all capsules from each row (each of the parent and M3 lines) were harvested for analysis of alkaloid content and profile. Capsules were weighed and, threshed to remove seed to produce poppy straw. The straw was then weighed and ground using a Retsch Grindomix GM 200 in preparation for alkaloid extraction. 2 g of the ground straw was placed in a plastic tube and suspended in 40 mL of extraction solution (consisting of 2% acetic acid and 10% ethanol in distilled water), then shaken on an orbital shaker for 90 minutes. 240 μL of extractant solution from each sample was filtered through a 0.45 μM Pall filter prior to the analysis of alkaloid content using a Waters Acquity ULPC® (Ultra High Performance Liquid Chromotography) system. Sample components were separated on a Waters Acquity UPLC BEH C18 Column (Part No. 186002352) by a gradient method using A. 2% acetic acid in water and B. acetonitrile (HPLC grade). The column temperature was maintained at 44 ° C. and the detector wavelength was set at 284 nm. Chromatographic peak areas were analysed and alkaloid content (% w/w) calculated using Empower software.

The results for each single replicate M3 line were compared to a mean value for the parent line (PW08-2308) included in the trial. Two other Papaver somniferum lines with a codeine chemotype, namely PW11-4063 and PW10-0149 derived from a breeding program, were included in the trial for comparison with the mutant M3 lines.

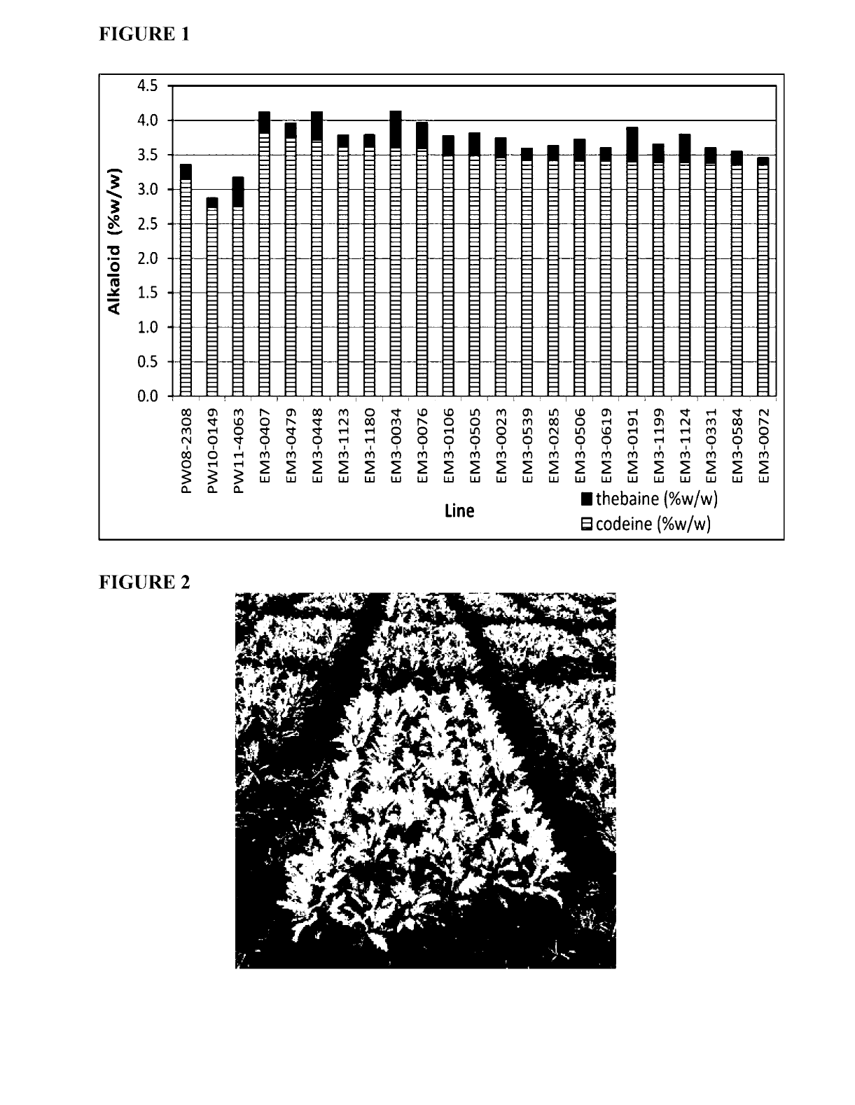

Of the 982 M3 lines screened, 80 M3 lines exhibited increased codeine content relative to the parent line (PW08-2308), and 139 M3 lines exhibited a thebaine/codeine ratio (T/C) lower than the parent line PW08-2308 (T/C of 0.06, that is a reduced amount of codeine relative to thebaine, the best lines being (EM3-0281, EM3-0538, EM3-0832 and EM3-0515, with a T/C of 0.01). No data was obtained for 68 M3 lines as a result of poor establishment in one section of the trial. Due to the high-vigour phenotype of the PW08-2308 parent line, M3 lines having mutations that affected plant health and vigour were very obvious. A total of 16 M3 lines were identified as showing very low vigour. Determined levels of codeine, thebaine, morphine and oripavine in selected M3 lines are shown in Table 1. A graph illustrating the codeine and thebaine content of the poppy straw from the best 20 codeine producing M3 lines is shown in FIG. 1

TABLE 1
Alkaloid content of poppy straw of selected Papaver somniferum M3 lines

| | | | | | | | | | Traits |
|---|---|---|---|---|---|---|---|---|---|
| | | | | | TOTAL | Thebaine/ | Codeine | TOTAL | improved |
| | codeine | morphine | oripavine | thebaine | MOCT | Codeine | % of | MOCT % of | relative to |
| Line | (% w/w) | (% w/w) | (% w/w) | (% w/w) | (% w/w) | ratio (T/C) | PW08-2308 | PW08-2308 | PW08-2308 |
| PW08-2308 | 3.15 | 0.00 | 0.00 | 0.20 | 3.36 | 0.06 | 100 | 100 | |
| PW10-0149 | 2.74 | 0.00 | 0.01 | 0.14 | 2.89 | 0.05 | 87 | 86 | |
| PW11-4063 | 2.76 | 0.00 | 0.00 | 0.42 | 3.17 | 0.15 | 87 | 94 | |
| EM3-0407 | 3.82 | 0.00 | 0.00 | 0.30 | 4.12 | 0.08 | 121 | 123 | codeine |
| EM3-0479 | 3.76 | 0.00 | 0.00 | 0.20 | 3.96 | 0.05 | 119 | 118 | codeine, |
| | | | | | | | | | T/C |
| EM3-0448 | 3.72 | 0.00 | 0.00 | 0.40 | 4.12 | 0.11 | 118 | 123 | codeine |
| EM3-1123 | 3.64 | 0.00 | 0.00 | 0.15 | 3.79 | 0.04 | 115 | 113 | codeine, |
| | | | | | | | | | T/C |
| EM3-1180 | 3.63 | 0.00 | 0.00 | 0.16 | 3.79 | 0.04 | 115 | 113 | codeine, |
| | | | | | | | | | T/C |
| EM3-0034 | 3.60 | 0.00 | 0.00 | 0.53 | 4.13 | 0.15 | 114 | 123 | codeine |
| EM3-0076 | 3.59 | 0.00 | 0.00 | 0.37 | 3.96 | 0.10 | 114 | 118 | codeine |
| EM3-0106 | 3.50 | 0.00 | 0.00 | 0.28 | 3.77 | 0.08 | 111 | 112 | codeine |
| EM3-0505 | 3.49 | 0.00 | 0.00 | 0.33 | 3.82 | 0.09 | 111 | 114 | codeine |
| EM3-0023 | 3.46 | 0.00 | 0.00 | 0.28 | 3.74 | 0.08 | 110 | 111 | codeine |
| EM3-0539 | 3.43 | 0.00 | 0.00 | 0.17 | 3.59 | 0.05 | 109 | 107 | codeine, |
| | | | | | | | | | T/C |
| EM3-0285 | 3.42 | 0.00 | 0.00 | 0.22 | 3.63 | 0.06 | 108 | 108 | codeine |
| EM3-0506 | 3.41 | 0.00 | 0.00 | 0.31 | 3.73 | 0.09 | 108 | 111 | codeine |
| EM3-0619 | 3.41 | 0.00 | 0.00 | 0.19 | 3.60 | 0.06 | 108 | 107 | codeine |
| EM3-0191 | 3.40 | 0.00 | 0.00 | 0.49 | 3.90 | 0.15 | 108 | 116 | codeine |
| EM3-1199 | 3.40 | 0.00 | 0.00 | 0.26 | 3.65 | 0.08 | 108 | 109 | codeine |
| EM3-1124 | 3.39 | 0.00 | 0.00 | 0.40 | 3.80 | 0.12 | 108 | 113 | codeine |
| EM3-0331 | 3.39 | 0.00 | 0.00 | 0.22 | 3.61 | 0.06 | 108 | 107 | codeine |
| EM3-0584 | 3.37 | 0.00 | 0.00 | 0.18 | 3.55 | 0.05 | 107 | 106 | codeine, |
| | | | | | | | | | T/C |
| EM3-0072 | 3.36 | 0.00 | 0.00 | 0.09 | 3.46 | 0.03 | 107 | 103 | codeine, |
| | | | | | | | | | T/C |
| EM3-1196 | 3.36 | 0.00 | 0.00 | 0.20 | 3.55 | 0.06 | 107 | 106 | codeine |
| EM3-0390 | 3.35 | 0.00 | 0.00 | 0.06 | 3.41 | 0.02 | 106 | 102 | codeine, |
| | | | | | | | | | T/C |
| EM3-0476 | 3.35 | 0.00 | 0.00 | 0.19 | 3.54 | 0.06 | 106 | 105 | codeine |
| EM3-0095 | 3.34 | 0.00 | 0.00 | 0.27 | 3.62 | 0.08 | 106 | 108 | codeine |
| EM3-1147 | 3.34 | 0.00 | 0.00 | 0.19 | 3.53 | 0.06 | 106 | 105 | codeine |
| EM3-1166 | 3.34 | 0.00 | 0.00 | 0.26 | 3.59 | 0.08 | 106 | 107 | codeine |
| EM3-0552 | 3.34 | 0.00 | 0.00 | 0.39 | 3.72 | 0.12 | 106 | 111 | codeine |
| EM3-0674 | 3.33 | 0.00 | 0.00 | 0.29 | 3.62 | 0.09 | 106 | 108 | codeine |
| EM3-0551 | 3.32 | 0.00 | 0.00 | 0.20 | 3.52 | 0.06 | 105 | 105 | codeine |
| EM3-1026 | 3.32 | 0.00 | 0.00 | 0.20 | 3.52 | 0.06 | 105 | 105 | codeine |
| EM3-1138 | 3.32 | 0.00 | 0.00 | 0.24 | 3.56 | 0.07 | 105 | 106 | codeine |
| EM3-0205 | 3.31 | 0.00 | 0.00 | 0.24 | 3.55 | 0.07 | 105 | 106 | codeine |
| EM3-0408 | 3.31 | 0.00 | 0.00 | 0.14 | 3.45 | 0.04 | 105 | 103 | codeine, |
| | | | | | | | | | T/C |
| EM3-1195 | 3.30 | 0.00 | 0.03 | 0.49 | 3.82 | 0.15 | 105 | 114 | codeine |
| EM3-0361 | 3.30 | 0.00 | 0.00 | 0.14 | 3.44 | 0.04 | 105 | 102 | codeine, |
| | | | | | | | | | T/C |
| EM3-1011 | 3.30 | 0.00 | 0.00 | 0.38 | 3.67 | 0.12 | 105 | 109 | codeine |
| EM3-0307 | 3.29 | 0.00 | 0.00 | 0.31 | 3.60 | 0.09 | 105 | 107 | codeine |
| EM3-1112 | 3.29 | 0.00 | 0.00 | 0.30 | 3.58 | 0.09 | 104 | 107 | codeine |
| EM3-0585 | 3.29 | 0.00 | 0.00 | 0.13 | 3.42 | 0.04 | 104 | 102 | codeine, |
| | | | | | | | | | T/C |
| EM3-1125 | 3.28 | 0.00 | 0.00 | 0.39 | 3.67 | 0.12 | 104 | 109 | codeine |
| EM3-1171 | 3.28 | 0.00 | 0.00 | 0.16 | 3.44 | 0.05 | 104 | 102 | codeine, |
| | | | | | | | | | T/C |
| EM3-0618 | 3.27 | 0.12 | 0.00 | 0.46 | 3.85 | 0.14 | 104 | 115 | codeine |
| EM3-0111 | 3.27 | 0.00 | 0.00 | 0.18 | 3.45 | 0.06 | 104 | 103 | codeine |
| EM3-0426 | 3.27 | 0.00 | 0.00 | 0.23 | 3.49 | 0.07 | 104 | 104 | codeine |
| EM3-1020 | 3.26 | 0.00 | 0.00 | 0.15 | 3.42 | 0.05 | 104 | 102 | codeine, |
| | | | | | | | | | T/C |
| EM3-1122 | 3.26 | 0.00 | 0.00 | 0.12 | 3.38 | 0.04 | 104 | 101 | codeine, |
| | | | | | | | | | T/C |
| EM3-1179 | 3.26 | 0.00 | 0.00 | 0.15 | 3.40 | 0.05 | 103 | 101 | codeine, |
| | | | | | | | | | T/C |
| EM3-1136 | 3.25 | 0.00 | 0.00 | 0.14 | 3.40 | 0.04 | 103 | 101 | codeine, |
| | | | | | | | | | T/C |
| EM3-0519 | 3.25 | 0.00 | 0.00 | 0.10 | 3.35 | 0.03 | 103 | 100 | codeine, |
| | | | | | | | | | T/C |
| EM3-0637 | 3.25 | 0.00 | 0.00 | 0.50 | 3.75 | 0.16 | 103 | 112 | codeine |
| EM3-0352 | 3.24 | 0.00 | 0.00 | 0.34 | 3.58 | 0.10 | 103 | 107 | codeine |
| EM3-0438 | 3.24 | 0.00 | 0.00 | 0.19 | 3.42 | 0.06 | 103 | 102 | codeine |
| EM3-0058 | 3.24 | 0.00 | 0.00 | 0.27 | 3.50 | 0.08 | 103 | 104 | codeine |
| EM3-0532 | 3.24 | 0.00 | 0.00 | 0.27 | 3.50 | 0.08 | 103 | 104 | codeine |
| EM3-0733 | 3.23 | 0.00 | 0.00 | 0.52 | 3.76 | 0.16 | 103 | 112 | codeine |
| EM3-0449 | 3.23 | 0.00 | 0.00 | 0.44 | 3.66 | 0.14 | 102 | 109 | codeine |
| EM3-0320 | 3.22 | 0.00 | 0.00 | 0.40 | 3.62 | 0.12 | 102 | 108 | codeine |
| EM3-0943 | 3.22 | 0.00 | 0.00 | 0.26 | 3.47 | 0.08 | 102 | 103 | codeine |
| EM3-0826 | 3.21 | 0.00 | 0.00 | 0.22 | 3.43 | 0.07 | 102 | 102 | codeine |
| EM3-1132 | 3.21 | 0.00 | 0.00 | 0.28 | 3.49 | 0.09 | 102 | 104 | codeine |
| EM3-0350 | 3.21 | 0.00 | 0.00 | 0.27 | 3.48 | 0.08 | 102 | 103 | codeine |
| EM3-0592 | 3.21 | 0.00 | 0.00 | 0.19 | 3.40 | 0.06 | 102 | 101 | codeine |
| EM3-1164 | 3.21 | 0.00 | 0.02 | 0.42 | 3.64 | 0.13 | 102 | 108 | codeine |
| EM3-0607 | 3.21 | 0.00 | 0.00 | 0.26 | 3.47 | 0.08 | 102 | 103 | codeine |
| EM3-1191 | 3.20 | 0.00 | 0.00 | 0.51 | 3.71 | 0.16 | 102 | 111 | codeine |
| EM3-0587 | 3.20 | 0.00 | 0.00 | 0.71 | 3.91 | 0.22 | 102 | 116 | codeine |
| EM3-1007 | 3.19 | 0.00 | 0.00 | 0.31 | 3.50 | 0.10 | 101 | 104 | codeine |
| EM3-0536 | 3.19 | 0.00 | 0.00 | 0.33 | 3.53 | 0.10 | 101 | 105 | codeine |
| EM3-0455 | 3.19 | 0.00 | 0.00 | 0.34 | 3.53 | 0.11 | 101 | 105 | codeine |
| EM3-0241 | 3.19 | 0.00 | 0.00 | 0.27 | 3.46 | 0.09 | 101 | 103 | codeine |
| EM3-0411 | 3.19 | 0.00 | 0.00 | 0.47 | 3.65 | 0.15 | 101 | 109 | codeine |
| EM3-0392 | 3.18 | 0.00 | 0.00 | 0.22 | 3.39 | 0.07 | 101 | 101 | codeine |
| EM3-1131 | 3.18 | 0.00 | 0.00 | 0.21 | 3.39 | 0.07 | 101 | 101 | codeine |
| EM3-0139 | 3.18 | 0.00 | 0.00 | 0.33 | 3.50 | 0.10 | 101 | 104 | codeine |
| EM3-0311 | 3.18 | 0.00 | 0.00 | 0.26 | 3.44 | 0.08 | 101 | 102 | codeine |
| EM3-0084 | 3.18 | 0.00 | 0.00 | 0.21 | 3.38 | 0.07 | 101 | 101 | codeine |
| EM3-1079 | 3.17 | 0.00 | 0.00 | 0.22 | 3.39 | 0.07 | 101 | 101 | codeine |
| EM3-0158 | 3.17 | 0.00 | 0.00 | 0.25 | 3.42 | 0.08 | 101 | 102 | codeine |
| EM3-0200 | 3.17 | 0.09 | 0.00 | 0.34 | 3.60 | 0.11 | 101 | 107 | codeine |
| EM3-1163 | 3.17 | 0.00 | 0.00 | 0.33 | 3.50 | 0.10 | 101 | 104 | codeine |
| EM3-0646 | 3.16 | 0.00 | 0.00 | 0.16 | 3.32 | 0.05 | 100 | 99 | T/C |
| EM3-1161 | 3.15 | 0.00 | 0.00 | 0.16 | 3.31 | 0.05 | 100 | 98 | T/C |
| EM3-0484 | 3.12 | 0.00 | 0.00 | 0.17 | 3.28 | 0.05 | 99 | 98 | T/C |
| EM3-1039 | 3.12 | 0.00 | 0.00 | 0.08 | 3.20 | 0.03 | 99 | 95 | T/C |
| EM3-0698 | 3.09 | 0.00 | 0.00 | 0.15 | 3.24 | 0.05 | 98 | 96 | T/C |
| EM3-1113 | 3.09 | 0.00 | 0.00 | 0.17 | 3.25 | 0.05 | 98 | 97 | T/C |
| EM3-1116 | 3.07 | 0.00 | 0.00 | 0.12 | 3.20 | 0.04 | 98 | 95 | T/C |
| EM3-1159 | 3.07 | 0.00 | 0.00 | 0.15 | 3.22 | 0.05 | 98 | 96 | T/C |
| EM3-0151 | 3.07 | 0.00 | 0.00 | 0.08 | 3.15 | 0.03 | 98 | 94 | T/C |
| EM3-1022 | 3.07 | 0.00 | 0.00 | 0.13 | 3.20 | 0.04 | 97 | 95 | T/C |
| EM3-1110 | 3.05 | 0.00 | 0.00 | 0.06 | 3.10 | 0.02 | 97 | 92 | T/C |
| EM3-1183 | 3.05 | 0.00 | 0.00 | 0.08 | 3.13 | 0.02 | 97 | 93 | T/C |
| EM3-0501 | 3.04 | 0.00 | 0.00 | 0.10 | 3.14 | 0.03 | 96 | 93 | T/C |
| EM3-0273 | 3.02 | 0.00 | 0.00 | 0.07 | 3.09 | 0.02 | 96 | 92 | T/C |
| EM3-0498 | 3.02 | 0.00 | 0.00 | 0.10 | 3.12 | 0.03 | 96 | 93 | T/C |
| EM3-0843 | 3.01 | 0.00 | 0.00 | 0.06 | 3.07 | 0.02 | 96 | 91 | T/C |
| EM3-1066 | 3.01 | 0.00 | 0.01 | 0.16 | 3.18 | 0.05 | 96 | 95 | T/C |
| EM3-0183 | 3.01 | 0.00 | 0.00 | 0.08 | 3.09 | 0.03 | 96 | 92 | T/C |
| EM3-0664 | 3.01 | 0.00 | 0.00 | 0.14 | 3.14 | 0.05 | 95 | 94 | T/C |
| EM3-0617 | 3.00 | 0.00 | 0.00 | 0.11 | 3.11 | 0.04 | 95 | 93 | T/C |
| EM3-0208 | 2.99 | 0.00 | 0.00 | 0.12 | 3.11 | 0.04 | 95 | 93 | T/C |
| EM3-0284 | 2.98 | 0.00 | 0.00 | 0.14 | 3.12 | 0.05 | 95 | 93 | T/C |
| EM3-0071 | 2.98 | 0.00 | 0.00 | 0.16 | 3.14 | 0.05 | 95 | 94 | T/C |
| EM3-0653 | 2.98 | 0.00 | 0.00 | 0.16 | 3.14 | 0.05 | 95 | 93 | T/C |
| EM3-0577 | 2.98 | 0.00 | 0.00 | 0.16 | 3.13 | 0.05 | 94 | 93 | T/C |
| EM3-1117 | 2.96 | 0.00 | 0.00 | 0.16 | 3.13 | 0.05 | 94 | 93 | T/C |
| EM3-0573 | 2.96 | 0.00 | 0.00 | 0.12 | 3.08 | 0.04 | 94 | 92 | T/C |
| EM3-0730 | 2.96 | 0.00 | 0.06 | 0.16 | 3.17 | 0.05 | 94 | 94 | T/C |
| EM3-1184 | 2.96 | 0.00 | 0.00 | 0.12 | 3.07 | 0.04 | 94 | 91 | T/C |
| EM3-0515 | 2.95 | 0.00 | 0.00 | 0.04 | 3.00 | 0.01 | 94 | 89 | T/C |
| EM3-0638 | 2.95 | 0.09 | 0.00 | 0.08 | 3.11 | 0.03 | 94 | 93 | T/C |
| EM3-0990 | 2.94 | 0.00 | 0.00 | 0.06 | 3.00 | 0.02 | 93 | 89 | T/C |
| EM3-0301 | 2.94 | 0.00 | 0.00 | 0.15 | 3.09 | 0.05 | 93 | 92 | T/C |
| EM3-0333 | 2.94 | 0.00 | 0.00 | 0.13 | 3.07 | 0.05 | 93 | 91 | T/C |
| EM3-0132 | 2.93 | 0.00 | 0.00 | 0.14 | 3.07 | 0.05 | 93 | 91 | T/C |
| EM3-0467 | 2.93 | 0.00 | 0.00 | 0.10 | 3.03 | 0.03 | 93 | 90 | T/C |
| EM3-0424 | 2.92 | 0.00 | 0.00 | 0.08 | 2.99 | 0.03 | 93 | 89 | T/C |
| EM3-0611 | 2.92 | 0.00 | 0.00 | 0.09 | 3.00 | 0.03 | 93 | 89 | T/C |
| EM3-0633 | 2.91 | 0.00 | 0.00 | 0.14 | 3.05 | 0.05 | 93 | 91 | T/C |
| EM3-1069 | 2.91 | 0.00 | 0.00 | 0.15 | 3.06 | 0.05 | 92 | 91 | T/C |
| EM3-0477 | 2.91 | 0.00 | 0.00 | 0.15 | 3.06 | 0.05 | 92 | 91 | T/C |
| EM3-0094 | 2.91 | 0.00 | 0.00 | 0.14 | 3.05 | 0.05 | 92 | 91 | T/C |
| EM3-1162 | 2.91 | 0.00 | 0.00 | 0.12 | 3.02 | 0.04 | 92 | 90 | T/C |
| EM3-0635 | 2.91 | 0.00 | 0.00 | 0.12 | 3.02 | 0.04 | 92 | 90 | T/C |
| EM3-0898 | 2.90 | 0.00 | 0.00 | 0.15 | 3.05 | 0.05 | 92 | 91 | T/C |
| EM3-0170 | 2.90 | 0.00 | 0.00 | 0.13 | 3.02 | 0.04 | 92 | 90 | T/C |
| EM3-0737 | 2.90 | 0.00 | 0.00 | 0.16 | 3.05 | 0.05 | 92 | 91 | T/C |
| EM3-0414 | 2.90 | 0.00 | 0.01 | 0.16 | 3.06 | 0.05 | 92 | 91 | T/C |
| EM3-0555 | 2.89 | 0.00 | 0.00 | 0.13 | 3.02 | 0.04 | 92 | 90 | T/C |
| EM3-0871 | 2.89 | 0.00 | 0.00 | 0.16 | 3.05 | 0.05 | 92 | 91 | T/C |
| EM3-1009 | 2.89 | 0.00 | 0.00 | 0.11 | 2.99 | 0.04 | 92 | 89 | T/C |
| EM3-0868 | 2.88 | 0.00 | 0.01 | 0.14 | 3.04 | 0.05 | 92 | 90 | T/C |
| EM3-0928 | 2.88 | 0.00 | 0.00 | 0.13 | 3.01 | 0.04 | 91 | 90 | T/C |
| EM3-0558 | 2.87 | 0.00 | 0.00 | 0.15 | 3.02 | 0.05 | 91 | 90 | T/C |
| EM3-0383 | 2.87 | 0.00 | 0.00 | 0.07 | 2.93 | 0.02 | 91 | 87 | T/C |
| EM3-0726 | 2.86 | 0.00 | 0.00 | 0.10 | 2.96 | 0.03 | 91 | 88 | T/C |
| EM3-0832 | 2.86 | 0.00 | 0.00 | 0.04 | 2.89 | 0.01 | 91 | 86 | T/C |
| EM3-0670 | 2.85 | 0.00 | 0.00 | 0.13 | 2.99 | 0.05 | 91 | 89 | T/C |
| EM3-0434 | 2.85 | 0.00 | 0.00 | 0.14 | 2.99 | 0.05 | 91 | 89 | T/C |
| EM3-0066 | 2.85 | 0.00 | 0.00 | 0.08 | 2.92 | 0.03 | 90 | 87 | T/C |
| EM3-0925 | 2.85 | 0.00 | 0.00 | 0.10 | 2.95 | 0.03 | 90 | 88 | T/C |
| EM3-1182 | 2.85 | 0.00 | 0.00 | 0.13 | 2.98 | 0.05 | 90 | 89 | T/C |
| EM3-1032 | 2.85 | 0.00 | 0.00 | 0.06 | 2.91 | 0.02 | 90 | 87 | T/C |
| EM3-1108 | 2.85 | 0.00 | 0.00 | 0.11 | 2.95 | 0.04 | 90 | 88 | T/C |
| EM3-1076 | 2.84 | 0.00 | 0.00 | 0.16 | 2.99 | 0.05 | 90 | 89 | T/C |
| EM3-0496 | 2.84 | 0.00 | 0.00 | 0.09 | 2.93 | 0.03 | 90 | 87 | T/C |
| EM3-0181 | 2.83 | 0.00 | 0.00 | 0.12 | 2.95 | 0.04 | 90 | 88 | T/C |
| EM3-1002 | 2.82 | 0.00 | 0.00 | 0.13 | 2.95 | 0.04 | 90 | 88 | T/C |
| EM3-0428 | 2.82 | 0.00 | 0.00 | 0.12 | 2.93 | 0.04 | 89 | 87 | T/C |
| EM3-0304 | 2.81 | 0.00 | 0.00 | 0.14 | 2.95 | 0.05 | 89 | 88 | T/C |
| EM3-1148 | 2.81 | 0.00 | 0.00 | 0.09 | 2.90 | 0.03 | 89 | 86 | T/C |
| EM3-0480 | 2.81 | 0.00 | 0.00 | 0.10 | 2.90 | 0.03 | 89 | 86 | T/C |
| EM3-0533 | 2.80 | 0.12 | 0.00 | 0.14 | 3.06 | 0.05 | 89 | 91 | T/C |
| EM3-0365 | 2.79 | 0.00 | 0.00 | 0.05 | 2.84 | 0.02 | 89 | 85 | T/C |
| EM3-0238 | 2.78 | 0.00 | 0.00 | 0.12 | 2.91 | 0.04 | 88 | 86 | T/C |
| EM3-1197 | 2.78 | 0.00 | 0.00 | 0.09 | 2.87 | 0.03 | 88 | 85 | T/C |
| EM3-0435 | 2.78 | 0.00 | 0.00 | 0.12 | 2.90 | 0.04 | 88 | 86 | T/C |
| EM3-0788 | 2.77 | 0.00 | 0.00 | 0.09 | 2.85 | 0.03 | 88 | 85 | T/C |
| EM3-0731 | 2.76 | 0.00 | 0.00 | 0.14 | 2.91 | 0.05 | 88 | 86 | T/C |
| EM3-0984 | 2.76 | 0.00 | 0.00 | 0.08 | 2.84 | 0.03 | 88 | 84 | T/C |
| EM3-1001 | 2.74 | 0.00 | 0.00 | 0.10 | 2.85 | 0.04 | 87 | 85 | T/C |
| EM3-0729 | 2.73 | 0.00 | 0.00 | 0.13 | 2.87 | 0.05 | 87 | 85 | T/C |
| EM3-0549 | 2.73 | 0.00 | 0.00 | 0.11 | 2.84 | 0.04 | 87 | 84 | T/C |
| EM3-0371 | 2.73 | 0.00 | 0.00 | 0.13 | 2.86 | 0.05 | 87 | 85 | T/C |
| EM3-0791 | 2.72 | 0.00 | 0.00 | 0.09 | 2.81 | 0.03 | 86 | 84 | T/C |
| EM3-0739 | 2.71 | 0.00 | 0.00 | 0.10 | 2.81 | 0.04 | 86 | 84 | T/C |
| EM3-0283 | 2.70 | 0.00 | 0.00 | 0.14 | 2.84 | 0.05 | 86 | 85 | T/C |
| EM3-1114 | 2.70 | 0.00 | 0.00 | 0.14 | 2.84 | 0.05 | 86 | 85 | T/C |
| EM3-0651 | 2.70 | 0.00 | 0.00 | 0.12 | 2.82 | 0.04 | 86 | 84 | T/C |
| EM3-0566 | 2.70 | 0.00 | 0.00 | 0.12 | 2.81 | 0.04 | 86 | 84 | T/C |
| EM3-0131 | 2.69 | 0.00 | 0.00 | 0.15 | 2.84 | 0.05 | 86 | 85 | T/C |
| EM3-0358 | 2.68 | 0.00 | 0.00 | 0.06 | 2.74 | 0.02 | 85 | 82 | T/C |
| EM3-0382 | 2.68 | 0.00 | 0.00 | 0.05 | 2.73 | 0.02 | 85 | 81 | T/C |
| EM3-1151 | 2.67 | 0.00 | 0.00 | 0.13 | 2.81 | 0.05 | 85 | 84 | T/C |
| EM3-0275 | 2.67 | 0.00 | 0.00 | 0.10 | 2.78 | 0.04 | 85 | 83 | T/C |
| EM3-0713 | 2.66 | 0.00 | 0.00 | 0.10 | 2.76 | 0.04 | 84 | 82 | T/C |
| EM3-1036 | 2.66 | 0.00 | 0.00 | 0.05 | 2.71 | 0.02 | 84 | 81 | T/C |
| EM3-0728 | 2.65 | 0.00 | 0.00 | 0.11 | 2.77 | 0.04 | 84 | 82 | T/C |
| EM3-0834 | 2.65 | 0.00 | 0.00 | 0.13 | 2.78 | 0.05 | 84 | 83 | T/C |
| EM3-0281 | 2.64 | 0.00 | 0.00 | 0.02 | 2.67 | 0.01 | 84 | 79 | T/C |
| EM3-0376 | 2.64 | 0.00 | 0.00 | 0.09 | 2.73 | 0.04 | 84 | 81 | T/C |
| EM3-0615 | 2.64 | 0.00 | 0.00 | 0.14 | 2.78 | 0.05 | 84 | 83 | T/C |
| EM3-0528 | 2.63 | 0.00 | 0.00 | 0.12 | 2.75 | 0.05 | 84 | 82 | T/C |
| EM3-0305 | 2.63 | 0.00 | 0.00 | 0.10 | 2.73 | 0.04 | 84 | 81 | T/C |
| EM3-0322 | 2.61 | 0.00 | 0.00 | 0.12 | 2.74 | 0.05 | 83 | 81 | T/C |
| EM3-0560 | 2.60 | 0.09 | 0.00 | 0.12 | 2.81 | 0.05 | 83 | 84 | T/C |
| EM3-0492 | 2.60 | 0.00 | 0.00 | 0.10 | 2.70 | 0.04 | 82 | 80 | T/C |
| EM3-0293 | 2.59 | 0.00 | 0.00 | 0.11 | 2.70 | 0.04 | 82 | 80 | T/C |
| EM3-0794 | 2.58 | 0.00 | 0.00 | 0.08 | 2.67 | 0.03 | 82 | 79 | T/C |
| EM3-0894 | 2.57 | 0.00 | 0.00 | 0.10 | 2.66 | 0.04 | 81 | 79 | T/C |
| EM3-0608 | 2.56 | 0.17 | 0.00 | 0.12 | 2.85 | 0.05 | 81 | 85 | T/C |
| EM3-0767 | 2.55 | 0.00 | 0.00 | 0.10 | 2.65 | 0.04 | 81 | 79 | T/C |
| EM3-1118 | 2.55 | 0.00 | 0.00 | 0.13 | 2.68 | 0.05 | 81 | 80 | T/C |
| EM3-0538 | 2.55 | 0.00 | 0.00 | 0.02 | 2.57 | 0.01 | 81 | 76 | T/C |
| EM3-0892 | 2.53 | 0.00 | 0.00 | 0.13 | 2.65 | 0.05 | 80 | 79 | T/C |
| EM3-0857 | 2.49 | 0.09 | 0.00 | 0.13 | 2.71 | 0.05 | 79 | 81 | T/C |
| EM3-0246 | 2.48 | 0.00 | 0.00 | 0.07 | 2.56 | 0.03 | 79 | 76 | T/C |
| EM3-0389 | 2.42 | 0.10 | 0.00 | 0.05 | 2.57 | 0.02 | 77 | 76 | T/C |
| EM3-0298 | 2.36 | 0.00 | 0.00 | 0.06 | 2.42 | 0.03 | 75 | 72 | T/C |
| EM3-0825 | 2.32 | 0.00 | 0.00 | 0.09 | 2.42 | 0.04 | 74 | 72 | T/C |
| EM3-0297 | 2.30 | 0.00 | 0.00 | 0.10 | 2.40 | 0.04 | 73 | 71 | T/C |
| EM3-0644 | 2.25 | 0.00 | 0.00 | 0.07 | 2.31 | 0.03 | 71 | 69 | T/C |
| EM3-1075 | 2.12 | 0.00 | 0.00 | 0.07 | 2.19 | 0.03 | 67 | 65 | T/C |

MOCT is the alkaloid combination of morphine, oripavine, codeine and thebaine.

2. M3 Disease resistance/phenotype screening trial

A disease resistance screening field trial (Hagley, Tasmania, Australia) was also conducted during the 2012/13 poppy growing season. A subset of 11 mutant lines identified on the basis of phenotype in the M2 (field) and M3 (planthouse) generations described above were selected for inclusion in this trial, including the ‘Tasman’ line EM4-0045 derived from parent line PW08-2308 by mutagenesis treatment with EMS as described in Example 1. The primary aim of this trial was to determine if the altered phenotypes resulted in any improvement in resistance to, or tolerance of, downy mildew (Peronospora meconosidis, previously known as Peronospora arborescens) infection. This trial was also an additional opportunity to further study plant phenotypes under field conditions.

M4 seed, bulked from multiple M3 plants of each line grown in the planthouse over the winter of 2013, was used to sow this trial. The trial was a randomized-complete-block-design with 3 blocks/replicates per line, each replicate was sown a 1.8 m wide×2.0 m long plot. The trial crop was not sprayed with any preventative or curative fungicides. Plants were allowed to mature and dry under field conditions, and 30 capsules were then harvested from each plot and combined for each individual replicate assay. Each 30-capsule-sample was threshed to remove seed to produce poppy straw, the straw was then weighed and ground, and the ground straw was extracted in 2% acetic acid and 10% ethanol in distilled water as described above. An aliquot of the extract was filtered prior to the UPLC analysis of alkaloid content and alkaloid profile as also described above. Data derived from the three replicate plots was analysed in Agrobase Generation II (Agronomix Software) using GLM (General Linear Model) analysis to produce an overall trial mean for each alkaloid in each M3 line.

There was no expectation that altered phenotypes of the mutant lines would result in an increase in alkaloid content, so the actual mutagenesis parent line (PW08-2308) was not been entered in the trial for comparison. However, ‘Tasman’ mutant line EM4-0045 surprisingly exhibited high-total alkaloid content and an altered leaf phenotype characterized by light-green leaves (see FIG. 2).

In a subsequent field trial (data not shown), the EM4-0045 light-green ‘Tasman’ mutant line exhibited a 5% increase in codeine relative to the parent line PW08-2308. Apart from the light green leaf colour, the phenotype of this line appears to be unchanged relative to the parent line, PW08-2308. As such, there appears to be no reason why the EM4-0045 mutant cannot be grown commercially.
3. Discussion

Various M2 plants were selected on the basis of altered phenotypes as outlined in Example 1. Some of these phenotypes were also present when M3 plants, derived from these M2 plants, were grown in various M3 screening trials. This demonstrates that these phenotypes are the result of stable, heritable, genetic changes, as the altered phenotypes were consistent across generations and environments.

An unexpected result from these trials was the identification of mutant lines that exhibited lighter coloured leaves (i.e., lighter green or yellow) and which contained high alkaloid content, suggesting a possible relationship between the altered leaf colour in these lines and alkaloid accumulation. These plants were not initially considered as being of interest in the M2 screen, and were harvested simply as a curiosity. Growing M3 seed from these lines in the greenhouse highlighted that these mutant phenotypes were both stable and heritable. Mutant line EM4-0045 in particular showed remarkably good vigour and its phenotype was largely unchanged relative to the parent line PW08-2308 other than the obvious difference in leaf colour. This line was included in the 2012/2013 disease resistance screening field trial to assess whether their phenotypic differences altered their level of resistance to downy mildew infection (Peronospora meconosidis). Whilst this proved to not to be the case and no apparent enhancement in protection against downy mildew infection was observed, the EM4-0045 (light-green leaf) ‘Tasman’ mutant exhibited very high codeine content with low thebaine contamination, a most striking and unexpected result. As a consequence, the EM4-0045 line was advanced to additional field trials for further assessment.

Overall, M3 Papaver somniferum mutant ‘Tasman’ lines were identified which showed an increase in codeine content relative to the parent line from which they were derived, as well as mutant ‘Tasman’ lines which also exhibited a reduction in thebaine content relative to codeine content. Given the nature of mutagenesis, the complex nature of alkaloid biosynthesis pathways in Papaver somniferum and the involvement of feedback mechanisms and the like as described above, it could not be predicted at the outset if any desirable changes in alkaloid content or profile were possible or if they could even be achieved via mutagenesis, which typically results in point mutations that either knock out or have a detrimental effect on gene function. Further, it could not be ascertained or predicted in advance as to whether any plants having altered phenotypes could be useful commercially.

### EXAMPLE 3

### Breeding Material Field Trial (Hagley, Tasmania, Australia)

A total of 82 mutant ‘Tasman’ M3 lines produced in Example 1 and their parent line, PW08-2308, were included in this field trial. The trial was conducted in a paddock (Hagley, Tasmania, Australia) during the 2013/14 poppy growing season, and was sown on 6 Sep. 2013. The trial was a randomized-complete-block design consisting of 2 blocks/replicates of each M3 line. Each replicate was sown in a single, 5 m long row within a 5 m×1.8 m plot that contained five individual rows. A visual assessment of plant phenotype was conducted prior to flowering. Plants were allowed to mature and dry under field conditions, and all of the capsules from plants in each row were then harvested (on 5 Feb. 2014) and pooled for analysis of seed and straw weight, and alkaloid content and profile. Capsules were weighed and threshed to remove seed to produce straw. The straw was weighed and ground, and the ground straw was extracted in 2% acetic acid and 10% ethanol in distilled water as described above. An aliquot of the extract was filtered prior to UPLC analysis of alkaloid content and alkaloid profile as also described above. The results for each M3 line were analysed in Agrobase Generation II (Agronomix Software) using GLM (General Linear Model) analysis and compared to the parent line, PW08-2308, and are set out below in Table 2.

TABLE 2
Alkaloid content of poppy straw of selected Papaver somniferum M3 lines

| | | | | Total | | Codeine |
|---|---|---|---|---|---|---|
| | Codeine (C) | Thebaine (T) | % C of | T + C | T/C | yield |
| Line | (% w/w) | (% w/w) | PW08-2308 | (% w/w) | ratio | (kg/Ha) |
| PW08-2308 | 3.5 | 0.4 | 100 | 3.9 | 0.11 | 29.0 |
| EM3-0352 | 4.2 | 0.5 | 120 | 4.6 | 0.11 | 43.9 |
| EM3-0006 | 4.1 | 0.1 | 119 | 4.2 | 0.02 | 49.8 |
| EM3-0106 | 4.1 | 0.3 | 118 | 4.4 | 0.07 | 34.5 |
| EM3-0587 | 4.1 | 0.4 | 116 | 4.5 | 0.11 | 39.4 |
| EM3-0455 | 4.0 | 0.3 | 116 | 4.3 | 0.07 | 39.8 |
| EM3-1026 | 4.0 | 0.2 | 116 | 4.2 | 0.04 | 70.6 |
| EM3-0426 | 4.0 | 0.1 | 116 | 4.2 | 0.04 | 26.3 |
| EM3-1123 | 4.0 | 0.3 | 115 | 4.3 | 0.07 | 41.3 |
| EM3-0505 | 4.0 | 0.4 | 115 | 4.4 | 0.09 | 58.2 |
| EM3-0350 | 4.0 | 0.3 | 114 | 4.3 | 0.07 | 60.3 |
| EM3-0766 | 3.9 | 0.5 | 113 | 4.4 | 0.13 | 21.7 |
| EM3-1125 | 3.9 | 0.3 | 113 | 4.2 | 0.04 | 37.3 |
| EM3-1136 | 3.9 | 0.2 | 112 | 4.1 | 0.06 | 46.9 |
| EM3-0479 | 3.9 | 0.2 | 112 | 4.1 | 0.05 | 56.7 |
| EM3-1164 | 3.9 | 0.2 | 112 | 4.1 | 0.05 | 49.4 |
| EM3-1179 | 3.9 | 0.1 | 112 | 4.1 | 0.02 | 39.0 |
| EM3-0733 | 3.9 | 0.3 | 111 | 4.2 | 0.08 | 69.6 |
| EM3-0408 | 3.9 | 0.1 | 111 | 4.0 | 0.04 | 47.3 |
| EM3-1045 | 3.9 | 0.8 | 111 | 4.6 | 0.20 | 34.2 |
| EM3-0407 | 3.8 | 0.3 | 110 | 4.2 | 0.08 | 49.6 |
| EM3-0686 | 3.8 | 0.3 | 109 | 4.2 | 0.09 | 61.5 |
| EM3-1124 | 3.8 | 0.1 | 109 | 3.9 | 0.04 | 2.9 |
| EM3-1138 | 3.8 | 0.1 | 109 | 3.9 | 0.04 | 17.3 |
| EM3-0056 | 3.8 | 0.2 | 108 | 4.1 | 0.06 | 30.3 |
| EM3-0826 | 3.8 | 0.2 | 108 | 4.0 | 0.05 | 43.2 |
| EM3-0285 | 3.8 | 0.2 | 108 | 4.0 | 0.07 | 58.4 |
| EM3-0331 | 3.8 | 0.4 | 108 | 4.3 | 0.10 | 57.3 |
| EM3-0023 | 3.8 | 0.2 | 108 | 4.1 | 0.06 | 51.2 |
| EM3-0411 | 3.8 | 0.4 | 108 | 4.1 | 0.10 | 41.3 |
| EM3-0191 | 3.7 | 0.3 | 107 | 4.3 | 0.05 | 29.0 |
| EM3-1196 | 3.7 | 0.3 | 107 | 4.1 | 0.09 | 21.5 |
| EM3-0592 | 3.7 | 0.4 | 107 | 4.1 | 0.10 | 46.3 |
| EM3-0072 | 3.7 | 0.3 | 107 | 4.1 | 0.08 | 45.1 |
| EM3-0111 | 3.7 | 0.3 | 107 | 4.0 | 0.07 | 28.2 |
| EM3-1122 | 3.7 | 0.2 | 107 | 3.9 | 0.05 | 30.1 |
| EM3-0018 | 3.7 | 0.3 | 107 | 4.0 | 0.04 | 45.0 |
| EM3-1112 | 3.7 | 0.4 | 106 | 4.1 | 0.12 | 25.1 |
| EM3-0448 | 3.7 | 0.5 | 106 | 4.3 | 0.13 | 35.0 |
| EM3-0607 | 3.7 | 0.3 | 106 | 4.0 | 0.09 | 72.0 |
| EM3-0674 | 3.7 | 0.4 | 106 | 4.1 | 0.10 | 43.2 |
| EM3-0637 | 3.7 | 0.2 | 106 | 3.9 | 0.05 | 24.7 |
| EM3-0076 | 3.7 | 0.4 | 105 | 4.1 | 0.11 | 40.6 |
| EM3-0519 | 3.7 | 0.3 | 105 | 4.0 | 0.08 | 64.2 |
| EM3-0476 | 3.7 | 0.1 | 105 | 3.8 | 0.02 | 47.9 |
| EM3-1011 | 3.7 | 0.4 | 105 | 4.1 | 0.11 | 57.7 |
| EM3-1007 | 3.7 | 0.2 | 105 | 3.9 | 0.06 | 22.0 |
| EM3-0619 | 3.6 | 0.3 | 104 | 4.0 | 0.09 | 42.4 |
| EM3-1132 | 3.6 | 0.4 | 104 | 4.1 | 0.12 | 37.6 |
| EM3-1166 | 3.6 | 0.3 | 103 | 3.9 | 0.09 | 16.2 |
| EM3-0585 | 3.6 | 0.4 | 103 | 4.0 | 0.11 | 22.0 |
| EM3-0390 | 3.6 | 0.2 | 103 | 3.8 | 0.06 | 45.3 |
| EM3-0241 | 3.6 | 0.3 | 103 | 4.0 | 0.09 | 53.4 |
| EM3-0584 | 3.6 | 0.3 | 103 | 3.9 | 0.09 | 34.0 |
| EM3-0058 | 3.6 | 0.5 | 102 | 4.1 | 0.08 | 60.6 |
| EM3-1020 | 3.5 | 0.2 | 102 | 3.8 | 0.07 | 46.8 |
| EM3-0552 | 3.5 | 0.3 | 101 | 4.0 | 0.09 | 30.0 |
| EM3-1180 | 3.5 | 0.2 | 101 | 3.9 | 0.07 | 37.3 |
| EM3-0539 | 3.5 | 0.2 | 101 | 3.8 | 0.07 | 23.7 |
| EM3-0764 | 3.5 | 0.9 | 101 | 4.4 | 0.13 | 58.7 |
| EM3-0034 | 3.5 | 0.5 | 101 | 4.0 | 0.16 | 36.0 |
| EM3-0551 | 3.5 | 0.4 | 101 | 3.9 | 0.06 | 47.7 |
| EM3-0307 | 3.5 | 0.2 | 100 | 3.8 | 0.06 | 34.0 |
| EM3-1131 | 3.5 | 0.3 | 100 | 3.7 | 0.07 | 71.7 |
| EM3-0205 | 3.5 | 0.3 | 100 | 3.8 | 0.08 | 39.4 |
| EM3-0801 | 3.5 | 0.5 | 99 | 4.0 | 0.14 | 32.9 |
| EM3-0361 | 3.4 | 0.2 | 99 | 3.9 | 0.04 | 58.4 |
| EM3-0506 | 3.4 | 0.4 | 99 | 3.9 | 0.13 | 49.0 |
| EM3-1147 | 3.4 | 0.4 | 98 | 3.8 | 0.06 | 46.5 |
| EM3-1198 | 3.4 | 0.9 | 97 | 4.3 | 0.27 | 30.4 |
| EM3-1171 | 3.3 | 0.4 | 95 | 3.7 | 0.11 | 28.5 |
| EM3-0095 | 3.3 | 0.5 | 94 | 3.9 | 0.14 | 27.7 |
| EM3-1199 | 3.3 | 0.8 | 94 | 4.1 | 0.28 | 50.1 |
| EM3-0320 | 3.3 | 0.7 | 94 | 4.0 | 0.23 | 43.9 |
| EM3-1191 | 3.2 | 0.5 | 92 | 3.9 | 0.19 | 27.2 |
| EM3-0449 | 3.2 | 0.7 | 91 | 3.8 | 0.22 | 23.3 |
| EM3-0532 | 3.1 | 0.4 | 90 | 3.6 | 0.12 | 21.5 |
| EM3-0934 | 3.1 | 0.4 | 88 | 3.7 | 0.14 | 33.5 |
| EM3-0643 | 2.9 | 0.5 | 84 | 3.8 | 0.19 | 21.0 |
| EM3-1195 | 2.9 | 0.3 | 84 | 3.8 | 0.11 | 25.1 |
| EM3-0438 | 2.8 | 0.2 | 81 | 3.8 | 0.06 | 25.8 |
| EM3-0618 | 2.8 | 0.6 | 81 | 3.6 | 0.10 | 38.7 |
| EM3-0536 | 2.5 | 0.6 | 71 | 3.1 | 0.32 | 30.7 |

Of the 82 lines tested, 61 showed improvement in codeine content relative to the PW08-2308 parent line. The best of these M3 lines, EM3-0352, exhibited a 20% improvement in codeine content. Interestingly, the next best M3 line, EM3-0006, which exhibited a 19% improvement in codeine content, also exhibited a very obvious light yellow-leaf phenotype. This result is consistent with the study described in Example 2 above which show high-alkaloid content in other M3 lines that exhibit a similar light leaf colour phenotype compared to the parent line PW08-2308. The co-occurrence of the lighter (light-green and light-yellow) leaf colour traits in several independent mutant lines suggests mutations in genes that can either directly, or indirectly, influence leaf colour as well as alkaloid content and alkaloid profile.

In addition to those lines that exhibited increases in codeine content, 56 M3 lines also exhibited lower thebaine content relative to codeine (T/C), than PW08-2308, which exhibited a T/C value of 0.11. The best of these M3 lines were EM3-0006 (light yellow leaf phenotype), EM3-1179 and EM3-0476, which all exhibited a T/C of 0.02. This suggests that, in the current trial location and season these lines were more efficient at converting thebaine through to codeine. When coupled with a high codeine content this low T/C trait is particularly valuable as it maximizes the amount of codeine available for extraction whilst reducing the level of thebaine that needs to be removed during factory processing.

### EXAMPLE 4

### Multi-Location Trials

Six field trials were sown in the 2014/15 poppy growing season with the purpose of identifying mutant Tasman lines with wide adaptability to different growing regions. The trials contained 18 entries including 3 commercially grown high codeine ‘Tasman’ Papaver somniferum producing lines, namely PW08-2308, PW11-4027 and PW11-4118. The trials were sown across a wide geographical distribution (Tunbridge, Cressy, Perth, Hagley, Latrobe, and North Motton), representing a diverse range of poppy growing regions in Tasmania, Australia. Each trial was sown within a poppy paddock and was treated the same as the surrounding crop in regards to herbicide, fertilizer, fungicide and irrigation treatments. Trial plants were not sprayed with any plant growth regulators.

Plants were allowed to mature and dry under field conditions. Immediately prior to the harvest of the surrounding commercial crop, all capsules from within a designated quadrat were hand-harvested from each trial plot. The harvested straw samples therefore consisted only of plant capsules and seed and contained no plant stems. All harvested material was stored for up to one month before being threshed to remove seed. These straw samples were ground, and the ground straw was extracted in 2% acetic acid and 10% ethanol in distilled water as described above. An aliquot of the extract was filtered prior to UPLC analysis of alkaloid content and alkaloid profile as also described above.

For each individual trial site, the alkaloid (% w/w) results for the three replicates of each M3 line were analysed in Agrobase Generation II (Agronomix Software) using “Alpha” analysis to produce a mean value for that M3 line at that trial site. An overall trial mean for each alkaloid (% w/w), from across the 6 trial sites, was then determined in Agrobase Generation II using the GxE Analysis function “ANOVA-combined RCBD: ENV. X ENTRY model. These overall trial means for codeine and thebaine content and related traits for these trials are shown in Table 3.

TABLE 3
Codeine and thebaine content of poppy straw from Papaver somniferum lines in 2014/2015 field trials

| | | | Total | | Codeine | Thebaine | Total | Codeine |
|---|---|---|---|---|---|---|---|---|
| | Codeine | Thebaine | MOCT | Thebaine/ | % of | % of | % of | yield % of |
| Line | (% w/w) | (% w/w) | (% w/w) | codeine | PW08-2308 | PW08-2308 | PW08-2308 | PW08-2308 |
| PW08-2308 | 3.50 | 0.35 | 3.89 | 0.09 | 100 | 100 | 100 | 100 |
| PW11-4027 | 3.52 | 0.29 | 3.85 | 0.09 | 101 | 83 | 99 | 96 |
| PW11-4118 | 3.57 | 0.29 | 3.90 | 0.10 | 102 | 81 | 100 | 94 |
| EM4-0045 | 4.01 | 0.20 | 4.21 | 0.06 | 115 | 56 | 108 | 103 |
| EM3-1204 | 3.88 | 0.21 | 4.07 | 0.06 | 111 | 61 | 105 | 102 |
| EM3-1217 | 3.83 | 0.31 | 4.17 | 0.09 | 109 | 90 | 107 | 103 |
| EM3-0056 | 3.73 | 0.41 | 4.16 | 0.12 | 107 | 116 | 107 | 93 |
| EM4-0270 | 3.64 | 0.30 | 3.95 | 0.08 | 104 | 85 | 102 | 89 |
| EM3-1203 | 3.44 | 0.56 | 4.02 | 0.18 | 98 | 159 | 103 | 93 |
| EM3-1213 | 3.39 | 0.33 | 3.77 | 0.07 | 97 | 94 | 97 | 78 |

Of the 7 mutant ‘Tasman’ lines evaluated in the present trials, 5 of the lines showed an improvement in codeine content (relative to the predominant commercial variety, PW08-2308) of between 4 and 15% on a % w/w basis. The best of these new lines was the light-green leaf mutant line EM4-0045, which exhibited the highest codeine content at 4 of the 6 trial sites.

Mutant lines EM3-1204 and EM3-1217 both showed high vigour and exhibited improvements in codeine content of 11% and 9% w/w, respectively, compared to PW08-2308.

Although thebaine is the precursor to codeine in the alkaloid synthesis pathway it is considered to be an undesirable ‘impurity” in poppy straw and latex for the production of codeine as it has negative implications for codeine extraction efficiency and thereby yield. A key criteria is, therefore, to minimize the amount of thebaine in poppy straw harvested for codeine production. The relevant measure of this trait, “tc”, is the amount of thebaine relative to the main alkaloid, codeine (tc=thebaine/codeine). In the 2014/15 season, the tc was 0.09 in PW08-2308, 0.10 in PW11-4118 and 0.09 in PW11-4027. In the present field trials, the mutant ‘Tasman’ lines with the highest codeine content (EM4-0045 and EM3-1204) both had a tc value of 0.06 in the trials. Moreover, both the EM4-0045 light green leaf mutant line and the EM3-1204 line displayed a substantially reduced level of thebaine relative to codeine compared to the parent line PW08-2308 from which they were derived.

Whilst of the 3 Papaver somniferum commercial lines both PW11-4118 and PW11-4027 exhibited lower thebaine to codeine levels than the PW08-2308, the 3 lines otherwise displayed very similar impurity profiles in the present trials. Other than the altered codeine and thebaine content of the poppy straw of the mutant ‘Tasman’ lines, those lines also showed very similar levels of impurity alkaloids the parent line PW08-2308 from which they were derived.

Papaver somniferum ‘Tasman’ lines generally exhibit even development, high vigour, and early flowering, a near absence of the twisted stem trait, and moderate resistance to down mildew (DM) infection. In the present field trials all but 1 (EM3-0056), exhibited very suitable phenotypes for being grown commercially.

In particular, of the 7 mutant lines included in these trials, EM3-1204, EM3-1217, and EM3-1203 all showed exceptional vigour. Clearly these lines do not contain mutations that have a negative visible impact on plant growth and development. The EM4-0045 (light-green leaf mutant) line in particular showed very good vigour across all trials, and a growth habit that was consistent with the commercially grown parent ‘Tasman’ line (PW08-2308) from which it was derived. This was unexpected given the light-green coloration of the leaves of these plants particularly during the vegetative stages of plant development. This colour difference between the mutant and the parent line was less obvious closer to flowering although it was still readily apparent in flower stems and capsules.

The observed increased codeine content and decreased t/c ratio of the EM4-0045 line relative to the parent line PW08-2308 is consistent with results for the EM4-0045 line from other field trials.

Given the above results it appears the mutation that results in the light green leaves in the EM4-0045 line does not cause negative pleiotropic effects on plant vigour and further, that this line does not contain other unrelated EMS-induced mutations that visibly affect plant growth, development and/or plant vigour.

### EXAMPLE 5

### Evaluation of Leaf Colour of Papaver Somniferum line EM4-0045

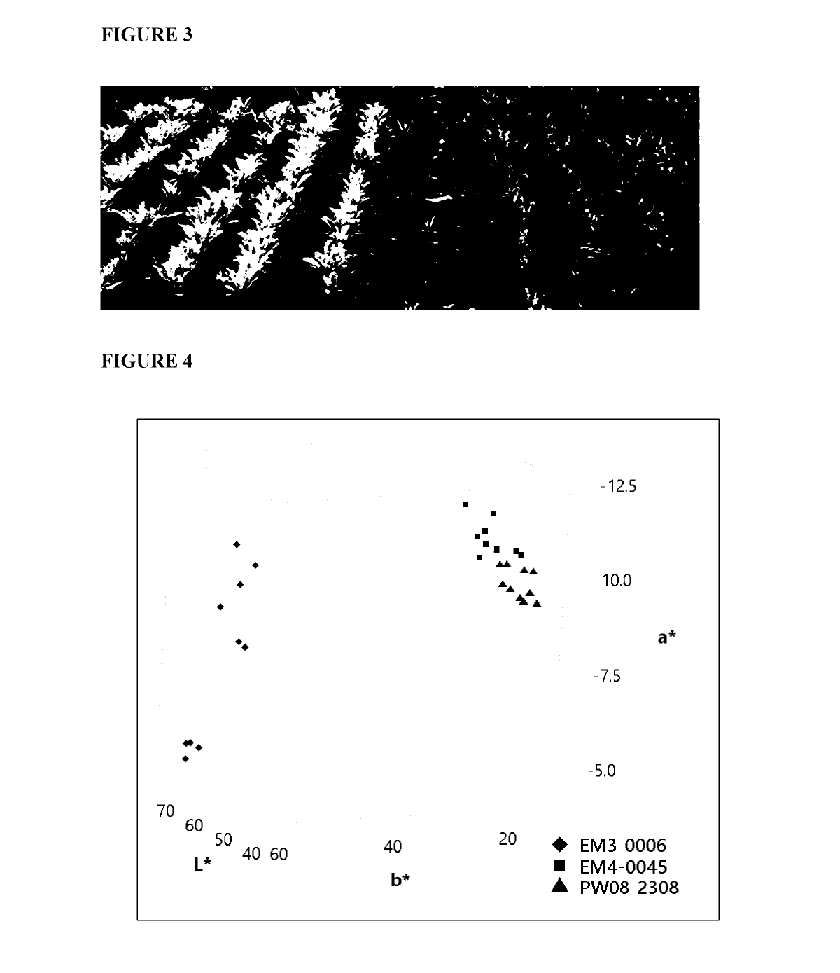

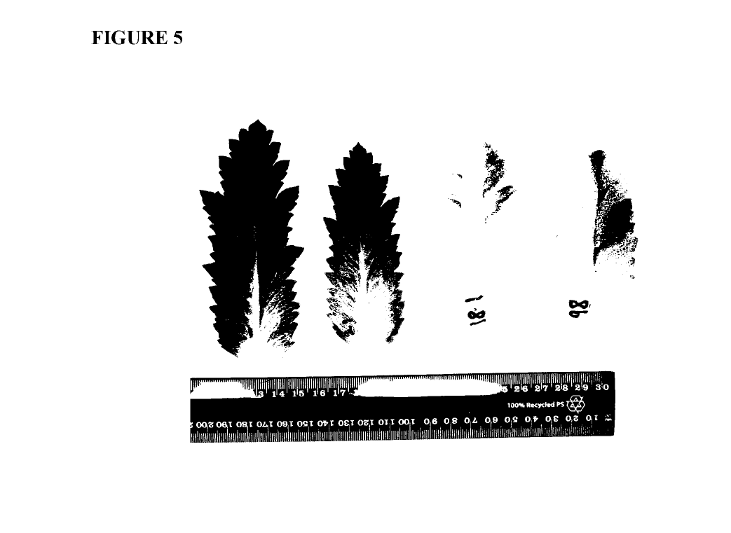

In addition to alkaloid differences, the EM4-0045 line exhibits a substantially lighter leaf and stem colour in comparison to the parental line, PW08-2308, and other typical commercially-grown P. somniferum ‘Tasman’ lines. This colour difference is highly marked in field grown plants as further illustrated in FIG. 3, which shows a field plot of EM4-0045 (left) growing next to a ‘Tasman’ line (right plot) exhibiting the typical darker green leaf phenotype in a field trial at Forest, Tasmania, in late 2016. To quantify this colour difference, spectrophotometer analysis was conducted on leaf tissues obtained from planthouse-grown plants. Also included in this analysis was a second high codeine line (EM3-0006) which has a light yellow leaf colour phenotype (see FIG. 5).
1. Plant Growing Conditions

Plants were grown in a planthouse at Tasmanian Alkaloids Pty Ltd, Westbury, Tasmania during the 2016 winter season. Seeds were sown on 23 Jun. 2016 in 20 cm diameter pots which contained a potting mix consisting of equal parts peat moss and composted pine bark. Once sown, seeds were covered with a thin (˜0.5 cm) layer of vermiculite and grown under an 18 hour light/8 hour dark photoperiod through use of supplemental lighting (high pressure sodium lamps; Horti Master greenPower, 600W 400v, E40). Automated irrigation and climate control systems were used to maintain pot moisture content at 30-40%volume and planthouse day and night temperatures at ˜20° C. and ˜15° C., respectively.

Following germination, seedlings were thinned to 5 plants per pot. A granular slow-release fertiliser was added to each pot on 9 Aug. 2016 (-1 g of Basacote Plus 3M; COMPO GmbH & Co. KG).
2. Tissue Sampling

A single leaf from each of ten individual EM4-0045, EM3-0006 and PW08-2308 plants was analysed by spectrophotometry. The plants from which the leaves were obtained were all healthy and viable with no symptoms of disease or nutrient deficiency. Leaves were abscised at the stem and immediately transferred to the

Chemical Research and Development laboratory at Tasmanian Alkaloids Pty Ltd, Westbury, Tasmania for analysis. As Papaver somniferum plants grow, older leaves towards the base of the plant begin to show visible signs of senescence. Therefore, younger leaves occurring towards the top of the stem which showed no visible signs of senescence and which were of a suitable size for spectrophotometer analysis (≥10 cm long×≥5 cm wide) were selected for analysis. For 19 out of 20 EM4-0045 and PW08-2308 plants, the fifth youngest leaf was sampled from plants (e.g., the leaf at the fifth leaf node down from the apical meristem). One PW08-2308 plant was sampled at the fourth youngest leaf due to the fifth leaf being damaged. In comparison to EM4-0045 and PW08-2308 lines, plants of the EM3-0006 line exhibit reduced vigour and delayed plant development. All plants were sampled on 18 Aug. 2016, 57 days after sowing. At this time, plants of EM4-0045 and PW08-2308 lines were 70-90 cm tall and in the early- to mid-hook developmental stage. In contrast, EM3-0006 line plants were shorter and in the running up stage.

The plants sampled for spectrophotometer analysis had been grown as part of a larger study involving two additional lines. In total, the experimental population comprised nine pots of each of five lines sown in a complete randomised block design. To obtain the 10 plants used for spectrophotometer analysis, a single plant was sampled from each of the nine PW08-2308, EM4-0045 and EM3-0006 pots, respectively, with each plant being randomly selected from within each pot. A tenth plant for each line was then obtained by sampling a second plant from one randomly selected pot.
3. Spectrophotometer Analysis

Spectrophotometry measurements were performed using a HunterLab UltraScan PRO spectrophotometer (Hunter Associates Laboratory, Virginia, USA). Reflectance (specular included) was measured on the upper-third region of each leaf (adaxial surface) using D65 illumination. Leaves were backed by a white tile and held against the 0.390″ port by the instrument's spring loaded clamp arm. Using reflectance measurements, CIE 1976 L*a*b* and tristmulus XYZ values were calculated using EasyMatch QC software (Hunter Associates Laboratory, Virginia, USA). To obtain the dominant wavelength values for each line, chromaticity x,y coordinates were firstly obtained by calculating CIE xyY values from XYZ values; where x=X/(X+Y+Z) and y=Y/(X+Y+Z). Each of these sample coordinates, in addition to the x,y coordinates of the D65 illuminant (x=0.31382, y=0.33100; CIE 1964 10°), were then plotted on a chromaticity graph drawn in Microsoft Excel 2010 using CIE 1964 10° chromaticity coordinates (0.1 nm interval values). A straight line was then drawn between the x,y coordinates of each line and the illuminant, respectively, with the line extrapolated out so as to intersect with the spectral locus λ_{D} (i.e., the dominant wavelength value).
4. Results

Individual spectrophotometer results (L*a*b* values) and mean dominant wavelength values for each line are shown in FIG. 4 and Table 4, respectively. As seen in the 3-dimensiona plot shown in FIG. 4, all samples of each line grouped within their respective clusters, and spectrophotometry analysis clearly differentiated the EM3-0006 line from both the EM4-0045 and PW08-2308 lines. As expected, this yellow leaf line (EM3-0006) was detected as being substantially lighter in colour (L*=66.92 vs. 43.82 to 45.25; where L*=0 is black and 100=‘brightest white’) and more yellow (b*=57.698 vs. 18.20 to 22.69; where greater positive values on the b* axis represent higher yellow colour values) in comparison to both PW08-2308 and EM4-0045 lines. These differences were reflected in dominant wavelength values, with the EM3-0006 line having a higher dominant wavelength of 568 nm (Table 4).

The spectrophotometer analysis of these planthouse-grown plants indicates that the leaves of the PW08-2308 and EM4-0045 lines are more similar in colour than when each is compared to the EM3-0006 line. Despite this, the PW08-2308 and EM4-0045 lines were separated on both the a* (range: PW08-2308 -9.36 to -10.38; EM4-0045-10.48 to −11.88) and b* (range: EM4-0045 19.88 to 26.26; PW08-2308 15.78 to 21.21) axes (see FIG. 4). Further, the PW08-2308 and EM4-0045 lines were detected as having dominant wavelengths of 560 nm and 561 nm, respectively (Table 4).

TABLE 4
Spectrophotometry results (mean values for each line) and dominant wavelength values

| | | | | CIE 1931 XYZ | | | CIE xyY | | Dominant |
|---|---|---|---|---|---|---|---|---|---|
| | CIE 1976 L*a*b* | | | colour space | | | colour space | | wavelengh^{a} |
| Line | L* | a* | b* | X | Y | Z | x | y | (nm) |
| PW08-2308 | 43.822 | −9.858 | 18.200 | 11.5824 | 13.7306 | 8.2301 | 0.3453 | 0.4093 | 560 |
| EM4-0045 | 45.250 | −11.040 | 22.690 | 12.3415 | 14.7912 | 7.6749 | 0.3546 | 0.4249 | 561 |
| EM3-0006 | 66.920 | −7.449 | 57.698 | 32.6997 | 36.6827 | 8.3704 | 0.4206 | 0.4718 | 568 |

^{a}Calculated using CIE 1964 10° standard observer and illuminant D65

The lighter leaf colour of EM3-006 plants compared to EM4-0045 plants when these plant lines are grown in a planthouse is shown in FIG. 5.
5. Discussion

When grown under common field conditions, the leaves of plants of the EM4-0045 line have a distinct light green-yellow appearance when compared to typical green Papaver somniferum poppy plants such as PW08-2308. Although the colour distinction between the planthouse-grown EM4-0045 and PW08-2308 line plants examined in this study was less pronounced than what is typically observed under field conditions, spectrophotometry analysis identified colour differences and detected the EM4-0045 line as being lighter and more yellow in colour, as evidenced by a dominant wavelength value of 561 nm for EM4-0045 in comparison to 560 nm for PW08-2308 (see Table 4).

The dominant wavelength values indicate that all three lines had leaf colours within the green-yellow colour spectrum. Thus, the three lines can be described as having green-yellow (PW08-2308), light green-yellow (EM4-045) and very light green-yellow (EM3-0006) leaf colours.

### EXAMPLE 6

### Light Green-Yellow Leaf Colour of Papaver Somniferum Line EM4-0045 is Associated with Reduced Leaf Chlorophyll and Carotenoid Pigment Content

The colour of planthouse-grown PW08-2308, EM4-0045 and EM3-0006 lines was quantified by spectrophotometry in Example 5 above. The physiological basis of the detected colour differences was examined in the present study by quantification of chlorophyll and carotenoid leaf pigments.
1 Tissue Sampling

The same populations of plants as described in above Example 5 were sampled for pigment analysis. The plants were sown in a complete randomized block design with each of three blocks containing one pot per line. Four plants within each pot were selected for sampling, resulting in a total of 12 plants per block, the tissues of which were combined to create ‘pooled’ samples. The EM3-0006 line had low establishment; eight plants in each of Blocks 1 and 2 and seven plants in Block 3, totaling 23 plants in all.

Briefly, the preparation of leaf tissue samples for pigment analysis involved the grinding of fresh tissues in a mortar and pestle under liquid nitrogen followed by freeze-drying and subsequent pigment extraction for HPLC analysis. The sixth youngest leaf was sampled from each plant with tissues being pooled within blocks for each line. Once harvested, pooled leaf tissues were segmented roughly (˜8 pieces per leaf), mixed, and then randomly selected until the desired fresh tissue weight for freeze-drying was obtained (˜11 g).

All plants were sampled when in the early-hook to mid-hook stage. For PW08-2308 and EM4-0045 lines sampling occurred 55-58 days after sowing. The slower development of the EM3-0006 line resulted in plants of this line being sampled approximately one week later (64 days after sowing). Leaves from all 23 sampled plants of the EM3-0006 line were combined into a single sample to obtain the required tissue weight for freeze-drying.

Once ground in liquid nitrogen, tissue samples were freeze-dried on a Christ Alpha 2-4-LD Plus freeze-drier. Four out of seven samples were degraded during freeze-drying and so were excluded from pigment analysis; the excluded samples comprised two EM4-0045 and two PW08-2308 replicates. A second set of leaves were therefore harvested from EM4-0045 and PW08-2308 plants post flowering (70 days after sowing). Here, one randomly selected plant from each of nine EM4-0045 and PW08-2308 pots were sampled and combined within lines, respectively. On this occasion, the fifth youngest leaf was sampled from each plant. Both samples were successfully freeze-dried.
2. HPLC Pigment Analysis

Following freeze-drying, 50 mg of ground freeze-dried leaf tissue was moistened with water (100-200 μl) and then extracted in an acetone:methanol (7:3) solution as described in Albert N W, Lewis D H, Zhang H, Irving L J, Jameson P E, Davies K M. (2009), Light-induced vegetative anthocyanin pigmentation in Petunia. J Exp Bot 60 (7):2191-2202. doi:10.1093/jxb/erp097. Samples were analysed using a Dionex Ultimate 3000 HPLC system with an Accucore RP C30 column. Absorbance was monitored using a photodiode array detector. Carotenoids and chlorophyll b were detected at 450 nm, while chlorophyll a and other chlorophyll derivatives were monitored at 430 nm. The levels of carotenoids were determined as β-carotene equivalents per gram of dry-weight (DW) of tissue. Chlorophyll a and b, respectively, were determined using chlorophyll a and b standard curves derived from a spinach extract. β-carotene and lutein were identified in the extracts by comparison of retention times and on-line spectral data with standard samples. Trans-β-carotene was purchased from Sigma Chemicals (St Louis, Missouri, U.S.A.). Other carotenoids (neochrome, α-carotene, violaxanthin and neoxanthin) were putatively identified by comparison with published retention times and spectral data for carotenoids present in the spinach extract.
3. Results

The number of samples in this study was reduced due to the degradation of samples during freeze-drying. Ultimately, a single sample for each of EM3-006, EM4-0045 and PW08-2308 was obtained at the hook timepoint. Furthermore, single samples were obtained for EM4-0045 and PW08-2308 at post flowering. Despite this, a marked decrease in leaf plant pigment content was clearly seen in the EM3-0006 line. This line contained less than 10% of the total chlorophyll present in the green-yellow progenitor line PW08-2308. A substantial reduction in carotenoid content was also observed in EM3-0006. Of interest, the lutein content of EM3-006 (341.6 μg^{-g} DW) exceeded the total chlorophyll a and chlorophyll b content of this line (317.1 μg^{-g} DW; see Table 5 below). The severe reduction of chlorophyll in EM3-0006 leaves may ‘unmask’ the yellow-coloured lutein pigment in the leaves of this line; thereby contributing to the very light green-yellow leaf colour observed.

The light green-yellow EM4-0045 line was also found to contain reduced levels of leaf chlorophyll; represented by ˜10-18% reductions in total chlorophyll content at both early hook and post flowering timepoints. A slight decrease in carotenoid pigments (˜4-5%) relative to the green-yellow progenitor line PW08-2308 was also observed for the EM4-0045 line.

The results are shown in Table 5. Values are presented in the table on a μg^{-g} dry-weight (DW) basis. Numbers in parentheses represent the percentage of total pigments (within each pigment class), and percentages relative to PW08-2308 values are calculated for lines within timepoints. H=hook, PF=post flowering and β-car=β-carotene.

TABLE 5
Chlorophyll and carotenoid content of planthouse-grown P. somniferum

| | | | | Chlorophylls | | | | | Carotenoids | | | | |
|---|---|---|---|---|---|---|---|---|---|---|---|---|---|
| | | | | | | | | % of | | | | | % of |
| | | Time- | Pool | | | a/b | | PW08- | | | | | PW08- |
| Line | Colour | point | size | CHL a | CHL b | ratio | Total | 2308 | Lutein | β-car | Other | Total | 2308 |
| EM3- | Very Light | H | 23 | 255.0 | 62.1 | 4.1 | 317.1 | 7.7 | 341.6 | 171.5 | 64.0 | 577.1 | 63.0 |
| 0006 | Green-yellow | | | (80.4) | (19.6) | | | | (59.2) | (29.7) | (11.1) | | |
| EM4- | Light | H | 10 | 2854.6 | 836.3 | 3.4 | 3690.9 | 89.7 | 592.9 | 199.9 | 92.8 | 885.6 | 96.7 |
| 0045 | Green-yellow | | | (77.3) | (22.7) | | | | (66.9) | (22.6) | (10.5) | | |
| PW08- | Green-yellow | H | 12 | 3192.0 | 924.8 | 3.5 | 4116.8 | | 611.4 | 209.3 | 95.5 | 916.2 | |
| 2308 | | | | (77.5) | (22.5) | | | | (66.7) | (22.8) | (10.4) | | |
| EM4- | Light | PF | 9 | 3296.9 | 963.6 | 3.4 | 4260.5 | 81.6 | 621.2 | 296.3 | 91.0 | 1008.5 | 94.6 |
| 0045 | Green-yellow | | | (77.4) | (22.6) | | | | (61.6) | (29.4) | (9.0) | | |
| PW08- | Green-yellow | PF | 9 | 4025.2 | 1199.2 | 3.4 | 5224.4 | | 716.3 | 247.6 | 102.2 | 1066.1 | |
| 2308 | | | | (77.0) | (23.0) | | | | (67.2) | (23.2) | (9.6) | | |

### EXAMPLE 7

### The Light Green-Yellow Colour Trait is Recessive and Associated with Capsule Alkaloid Content

A planthouse-grown F2 population was generated to examine the inheritance pattern of the light green-yellow colour trait. The Tasman light green-yellow line EM4-0045 and another P. somniferum line shown to be pure-breeding for the typical wild-type, green leaf phenotype (designated as the “second parent” line) were used as parents in the F2 pedigree. Following the initial EM4-0045×second parent cross, the resulting F1 line (X15-0260) was self-pollinated to produce the F2 generation (PH16-2253 line).
1. Plant Growing Conditions

Eight pots of each parental line (EM4-0045 and the second parent line), the F1 (X15-0260) and 50 pots of the F2 generation (PH16-2253) plants were grown in a planthouse at Tasmanian Alkaloids Pty Ltd (Westbury, Tasmania, Australia) during the 2017 winter season. Eight pots of the Tasman green line PW08-2308 from which the EM4-0045 line was derived was also planted as a control. Sowing occurred on 20 Apr. 2017 with seeds sown in 20 cm diameter pots. The potting mixture (per m³) comprised composted pine bark (800 L), sand (100 L), peat moss (100 L), dolomite lime (3 kg), hydrated lime (3 kg) and rock gypsum (1 kg). Once sown, seeds were covered with a thin (˜0.5 cm) layer of vermiculite and grown under an 18 hour light/8 hour dark photoperiod through use of supplemental lighting (high pressure sodium lamps; Horti Master greenPower, 600 W 400v, E40). Automated climate control systems were used to maintain planthouse day and night temperatures at ˜20° C. and ˜15° C., respectively. Soil moisture content was maintained at 30-40% volume via an automated fertigation system which also supplied plant nutrients.
2. Assessment of the Colour Trait

Plants within the population were visually assessed for leaf colour on 12 May 2017 (22 days after sowing) when seedlings were at the 4-6 true leaf stage (i.e., a foliage leaf of the plant as opposed to the cotyledonary leaves). All plants of both the PW08-2308 and second parent lines exhibited wild-type green colour phenotypes. As expected, all plants of the light green-yellow line EM4-0045 had light green-yellow coloured leaves. All plants of the F1 line X15-0260 were green, suggesting that the light green-yellow colour phenotype is a recessive trait. Once assessed for colour phenotypes, pots of the PW08-2308, second parent, EM4-0045 and X15-0260 lines were thinned to six plants per pot.

The F2 population (50 pots) contained a total 411 plants. Owing to variable sowing rates the number of plants per pot ranged from between 3 to 21 (average number of plants per pot =8.22, mode =10). Following an initial assessment of the F2 population for colour phenotype on 12 May 2017 a second assessment was undertaken (16 May 2017) to double-check colour phenotypes within the segregating F2 population. Two abnormally small plants were scored as ‘unsure’ with all other plants being assessed as either green (305) or light green-yellow (104). The observed green and light green-yellow frequencies fitted a 3:1 segregation ratio (x² 0.0399, df=1, P=0.8416; where observed N=409 and expected green and light green-yellow plant frequencies were 306.75 and 102.25, respectively), indicating that the light green-yellow leaf trait of the EM4-0045 line is inherited as a single-gene recessive trait.

Pots of the F2 population were thinned to six plants per pot following the second phenotype assessment. The low sowing rate in some pots resulted in eight pots having less than six plants per pot. For example, a single pot contained three plants, two pots contained four plants per pot and five pots contained five plants per pot.
3. Capsule Alkaloid Content

Plants were grown to maturity and allowed to dry naturally. Very few plants developed secondary branches/capsules with nearly all plants within the population being single-stemmed plants. To prevent outcrossing with neighboring poppy lines, all plants were ‘bagged selfed’. This involved placing a small paper bag over the unopened flower bud when in the ‘late hook’ or ‘upright bud’ stage and securing the bag with a plastic tie. This bagging method prevents pollen dispersal and outcrossing of the bagged plant yet still allows for self-pollination to occur.

Following plant desiccation, capsules were abscised from plants at the position directly below the peduncle. The primary capsule was harvested from 15×PW08-2308, 15×EM4-0045, 20×“Green-yellow” F2 and 20×“Light green-yellow” F2 individual plants, respectively. Seeds were removed before oven-drying capsules at 65° C. for 12 hours to remove any residual moisture content and to standardize moisture content across samples. Individual capsules were then ground to a consistent particle size using an electric coffee/spice grinder. The average weight of dried ground capsules was 1.20 g (range 0.39 g to 2.55 g). The whole, ground capsule material was used for individual capsule alkaloid extractions. For five samples where the oven-dried capsule weight exceeded two grams, a 2.00 g subsample was used for alkaloid extraction.

Capsule alkaloids were extracted in a 2% acetic acid and 10% ethanol solution using a 2.00 g tissue to 40 mL extractant ratio, which was scaled accordingly for the variable capsule weights. Samples were shaken for 90 minutes on a Ratek orbital shaker before transferring a 240 μL aliquot of each sample to a 96-well Pall filter plate (GHP 0.2 μm). Samples were then filtered under vacuum into a 96-well collection plate for UPLC analysis utilising the protocol described in Example 2 above.
4. Capsule Alkaloid Results

Capsular alkaloid content was quantified in the light green-yellow parental line EM4-0045, the progenitor Tasman line PW08-2308, and two phenotypic classes of the F2 generation (F2 Green-yellow and F2 Light Green-yellow).

As seen in Table 6, the light green-yellow line EM4-0045 was found to contain a higher mean codeine content and lower mean thebaine content on a percent dry weight basis (% w/w) when compared to its green-yellow progenitor line PW08-2308. The lower mean thebaine to codeine ratio (T/C) of the EM4-0045 line was found to be substantially lower than that of the wild-type PW08-2308 line (0.013% w/w v 0.061% w/w).

Further, the F2 Light Green-yellow plants were found to have a higher mean codeine content (3.342% w/w v 3.212% w/w), lower mean thebaine content (0.516% w/w v 0.727% w/w) and a lower mean T/C ratio (0.164 v 0.243; Table 6) in comparison to F2 Green-yellow plants.

The alkaloid values shown in Table 6 are line/class means. Within each pair-wise comparison, the alkaloid content of the Light Green-yellow line/classes are shown as a percentage of the Green-yellow line/classes, respectively.

TABLE 6
Capsule alkaloid results obtained from the greenhouse-grown F2 population.

| Generation | Parental-control pair-wise comparison | | | F2 generation pair-wise comparison | | |
|---|---|---|---|---|---|---|
| and comparison | | | % of Green- | F2 | F2 Light | % of Green- |
| Line or class | PW08-2308 | EM4-0045 | yellow line | Green-yellow | Green-yellow | yellow line |
| N | 15 | 15 | | 20 | 20 | |
| Leaf colour | Green-yellow | Light | | Green-yellow | Light | |
| | | green-yellow | | | green-yellow | |
| Alkaloid | | | | | | |
| trait | | | | | | |
| Codeine (C) | 3.669 | 3.838 | 104.6 | 3.212 | 3.342 | 104.0 |
| % w/w | | | | | | |
| Thebaine (T) | 0.243 | 0.052 | 21.4 | 0.727 | 0.516 | 70.9 |
| % w/w | | | | | | |
| T/C ratio | 0.061 | 0.013 | 21.3 | 0.243 | 0.164 | 67.5 |

In summary, analysis of segregation patterns in the F2 generation has shown that the light green-yellow leaf colour phenotype of the EM4-0045 line is genetically controlled by a single gene, and that the light green-yellow allele is recessive to the wild-type allele responsible for the normal green-yellow leaf colour phenotype. The results also show that the light green-yellow leaf colour trait is associated with beneficial alkaloid traits in poppy straw on a dry weight basis of the straw, namely an increase in codeine content by weight, a decrease in thebaine content by weight, and an overall decrease in the ratio of thebaine to codeine (T/C) by weight in the poppy straw.

While the foregoing specification teaches the principles of the present invention, with examples provided for the purpose of illustration, it will be understood that the practice of the invention encompasses variations, adaptations and/or modifications as come within the scope of the following claims and their equivalents.

1. An isolated plant of Papaver somniferum having a stably heritable codeine chemotype in which codeine is the predominant alkaloid and which upon harvesting of its poppy capsules will yield a poppy straw having a codeine content of greater than 2.0% on a w/w basis of the poppy straw, wherein the plant is a plant of a Papaver somniferum plant line having an introduced trait for a lightened leaf colour and the trait is associated with a higher codeine content by weight and/or a lower thebaine to codeine ratio in the poppy straw of the plant than in the absence of the trait.

2. The plant of claim 1, wherein the codeine chemotype of the plant is characterised by higher codeine content by weight than a combined morphine and thebaine content of the plant.

3. The plant of claim 1, wherein the trait is associated with a higher codeine content by weight and a lower thebaine to codeine ratio in the poppy straw than in the absence of the trait.

4-5. (canceled)

6. The plant according to claim 1, wherein the colour of the leaves of the viable said plant is in the green-yellow colour spectrum and has a dominant reflective wavelength in a range of from about 561 nm to about 568 nm as determined by reflective spectrophotometry of the adaxial leaf surface using D65 illumination and CIE 1964 10° standard observer coordinates.

7. The plant according to claim 1, wherein the trait is associated with a reduced leaf pigment content comprising a reduced level of at least one of chlorophylls and carotenoids in the leaves of the plant.

8-11. (canceled)

12. A plant according to claim 1, wherein the poppy straw has a codeine content
of about 2.5% or greater on a w/w basis of the poppy straw.

13-16. (canceled)

17. The plant according to claim 1, wherein the poppy straw has a thebaine to codeine ratio of about 15% or less on a w/w basis.

18-20. (canceled)

21. The plant according to claim 1, wherein there is substantially no morphine and substantially no oripavine in the poppy straw.

22. (canceled)

23. The plant of claim 1 wherein the trait is a single-gene recessive trait.

2-30. (canceled)

31. An isolated viable plant of Papaver somniferum having a stably heritable codeine chemotype in which codeine is the predominant alkaloid and which upon harvesting of its poppy capsules yields a poppy straw having a codeine content of greater than 2.0% on a w/w basis of the poppy straw, wherein the leaves of the plant are predominantly of a colour exhibiting a dominant wavelength in a range of from about 561 nm to about 568 nm as determined by reflective spectrophotometry on the adaxial leaf surface using D65 illumination and CIE 1964 10° standard observer coordinates.

32. The plant according to claim 31, wherein the plant is an

33. (canceled)

34. A method for producing a plant of Papaver somniferum having a stably heritable codeine chemotype characterised by higher codeine content by weight than a combined morphine and thebaine content, or a descendent plant thereof, the method comprising the steps of:
a) exposing at least one poppy seed of a Papaver somniferum parent plant to a mutagenizing agent, the parent plant being a plant which upon the harvesting of its poppy capsules will yield a poppy straw having codeine constituting about 40% by weight or greater of an alkaloid combination comprising morphine, codeine, thebaine and oripavine;
b) growing the at least one poppy seed exposed to the mutagenizing agent to produce one or more further plants, optionally through one or more self-fertilised generations;
wherein upon harvesting of its poppy capsules, said one or more further plants yields a poppy straw having a higher absolute codeine content and/or a lower thebaine to codeine ratio by weight, than the poppy straw of the parent plant, and further wherein the one or more further plants has a trait for a lightened leaf colour whereby the adaxial leaf surface of the leaves of the one or more further plants are a colour that is visually lighter relative to the leaves of the parent plant.

35-50. (canceled)

51. The method according to claim 34, wherein the colour of the adaxial leaf surface of leaves of the one or more further plant is in the green-yellow colour spectrum and has a dominant wavelength in a range of from about 561 nm to about 568 nm as determined by reflective spectrophotometry on the adaxial surface of the leaf using D65 illumination and CIE 1964 10° standard observer coordinates.

52-55. (canceled)

56. A poppy straw obtained from the plant of claim 1.

57. A concentrate of poppy straw of the poppy straw of claim 56.

58. A latex from immature poppy capsules of the plant according to claim 1.

59. An opium, a codeine, or a preparation of codeine claim 1.

60. (canceled)

61. A seed, a plant cell, or a plant root of a plant of claim 1.

62. (canceled)

63. A seed of claim 62, deposited as NCIMB deposit NCIMB 42630.

64. A plant of claim 1, deposited as NCIMB deposit NCIMB 42630, or a descendent plant thereof.

65. (canceled)
